(19) United States
(12) Patent Application Publication
(10) Pub. No.: US 2009/0275038 A1
(43) Pub. Date: Nov. 5, 2009

(54) METHOD AND APPARATUS FOR FORENSIC SCREENING

(75) Inventor: Timothy A. Hodge, Eads, TN (US)

Correspondence Address: BUTLER, SNOW, O'MARA, STEVENS & CANNADA PLLC, 6075 POPLAR AVENUE, SUITE 500, MEMPHIS, TN, 38119, US

(73) Assignee: TransnetYX, Inc., Cordova, TN (US)

(21) Appl. No.: 12/419,978

(22) Filed: Apr. 7, 2009

Related U.S. Application Data
(60) Provisional application No. 61/043,019, filed on Apr. 7, 2008.

(51) Int. Cl. G06Q 50/00; G06Q 10/00; C12Q 1/68; G06F 19/00

(52) U.S. Cl. 435/6; 705/8; 702/19

(57) ABSTRACT

This invention relates to methods and system for forensic screening. More specifically, this invention relates to various methods to detect or screen for at least one designated genetic sequences in a plurality of biological sample 40 using a template 30 in conjunction with a laboratory management software 24 to automatically control a workstation and to automatically rescreen samples 40 to achieve a high level of accuracy while tracking the samples 40 for chain of custody purposes.

Related Applications This application claims the benefit of U.S. Ser. No. 61/043,019 under 35 U.S.C. Sec. 119(e) (hereby specifically incorporated by reference in its entirety).

### BACKGROUND OF THE INVENTION

The present invention relates to field of processing physical data so that forensic screening of biological sample are sufficiently reliable to be admissible in a court of law and that the physical data can be processed rapidly so that it is useful in a criminal investigation.

Microsatellite loci, generally known in forensic applications as Short Tandem Repeat (STR) loci, are widely used for forensic identification and relatedness testing, and are the predominant genetic markers in this area of application. In forensic identification cases, the goal is typically to link an individual with a sample of blood, semen, sputum, bone, vaginal secretions, bodily excretions, cerebral spinal fluid, fingernails, epithelial cells, lymph, biological tissue or hair taken from a crime scene or victim. This information can generate linkages between two people or individuals and particular locations. Relatedness testing in criminal work may involve investigating paternity in order to establish rape or incest. Another application involves linking DNA sample with relatives of a missing person.

Microsatellite screening, in both research laboratories and crime laboratories, is a process where many of the steps are currently done manually. Manual manipulation of sample is a large source of error in the laboratory environment. The present manual system is time-consuming and can provide variable results depending on the laboratory and even depending on skill of laboratory workers. Manual nucleic acid isolations, PCR amplification, amplicon quantification and capillary electrophoresis of up to 30 sample can take most laboratories day, weeks, months and even years to process depending on the priority assigned to the type of sample.

### SUMMARY OF THE INVENTION

In this method a workstation is automatically directed by a template, while recording a sample identifier at each step of the screening process. This computer readable sample identifier is associated with the process steps and allows validation of the chain of custody of the sample. If nonconforming data is detected, a work list is generated and the workstation is directed to rescreen the sample that does not conform to the template. In this way the process can be automated, but is still sufficiently reliable for the results to be used in court. Additionally, the method operating according to the features described herein can provide screening results to a crime laboratory from the vendor laboratory within eight hours of receiving the biological sample while maintaining the chain of custody.

The application provides a method for obtaining measurement data of one or more physical samples, such as a piece of clothing, swabs, blood cards, finger nails etc. A vendor lab or analyzing lab receives electronic data from a user which can be a forensic lab, a crime lab or any other source of samples. The electronic data is provided in a form which allows them to be directly and immediately used by an order manager, by a process controller, or by a LIMS data base of the vendor lab. It is not necessary that the data can be used by the entire aforementioned unit but it is sufficient if only one or two of the order manager, the process controller 26, or the LIMS data base can use the electronic data. The order manager, the process controller 26, or the LIMS data base can be provided as a software which is running on one or more computers at the vendor lab.

The vendor lab receives a physical sample which corresponds to the electronic data and the process controller uses at least part of the received electronic data for automatically controlling the handling of the samples by operating units of a workstation of the vendor lab. Such operating units can include a bar code scanner, a lysis station, an accessing station, an isolation station, quantification and normalisation stations, amplification stations as well as CE plate building and detection stations. The data which is used by the process controller 26 is usually the received electronic data directly itself. The data can also be derived from the originally received electronic data by automatically transforming it according to a mathematical rule, such as normalization or transformation.

The vendor lab then generates measurement data by evaluating the physical samples in at least one operating unit of the workstation and outputs the measurement data, e.g. on paper, on a screen, or by sending an email with the measurement result is electronic form.

In the general case, the data can be provided by the crime lab e.g. in the form of a data template or in other data structures but it is also possible to provide a data template by the vendor lab. The structure of the data template may then be derived from the received electronic data and the template is then populated with at least part of the received electronic data. The data template can also be provided at the user lab for populating it with the received data from the crime lab. The structure of the data template can be individually adapted to the specific crime/user lab and then populated or filled with the electronic data which is provided by the user lab. Different user labs can then have different templates which do also have a different structure.

In a further embodiment of the application, the data template can be generated in a time slot between receiving a first part of the electronic data and a second part of the electronic data. This applies especially when a user lab sends a first part of the data with an electronic request, upon which the vendor lab generates the individual data template with individual structure for the specific user lab. After that, the crime lab uses only the second part or also the first part of the electronic data for populating the data template which has been generated before.

It is possible to use electronic data which is at least partly received from a LIMS at the crime/user lab but the electronic data can also at least partly be received from a webpage or homepage with possibly limited access which is provided by the vendor lab. It is not always necessary to provide two parts of electronic data, rather than sending all of the data including designations in two parts but when the first part of electronic data is used for creating the data template, it would at least be in place before the physical samples arrive at the vendor lab. The second part of electronic data may then be sent just before or after arrival of the physical samples. The data template could also be created at the time the samples arrive. The template also need not be populated by the crime lab in one embodiment; rather the vendor lab could populate the template on behalf of the crime laboratory.

The data template can include the specifications that the data is to meet in order to be acceptable to the specific crime laboratory. The template is then the defined acceptable data parameters for that specific laboratory. The data produced by the workstation is compared against the parameters of the template, if the created data is falls outside or is otherwise non-conforming, any of the multiple parameters defined in the template, then a Corrective Action step for that particular parameter, which is defined in the template in case that specific error occurs, which defines what is to happen to that specific sample. Based on the Corrective Action defined in the template LIMS writes a Corrective Action worklist to re-process the data to make it conform to the template.

In a specific embodiment of the application a LIMS data Template is used, wherein LIMS is tracking at every step and automated process controller handling and controlling the work station and its components. This results in a mechanized anti-tampering device or vendor laboratory with a device for receiving electronic data from a sender or crime laboratory which is at a location which is physically separate from the analysis laboratory, with a device for receiving a physical sample which corresponds to the electronic data, the analysis laboratory comprising a process controller device which uses at least part of the received electronic data for automatically controlling the handling of the samples by operating units of a workstation device, the analysis laboratory further generating measurement data by evaluating the physical samples in at least one operating unit of the workstation device and outputting the measurement data.

The anti-tampering device or laboratory provides court-proof data and avoids common errors that happen in a laboratory as follows. Samples can become contaminated from other samples or workers in the typical laboratory setting. The samples can be mixed up with one another. The chain-of-custody can be broken by lack of documentation. The data can be of poor quality when high quality of data is achievable based on the techniques, skill sets and experience of each employee in typical laboratory. The embodiments avoid such mistakes.

The results are often sent to the crime/user lab as numeric data, e.g. in the form of a visual illustration. Often, a FSA or CMF file is sent out that can be imported into a software for data review. Once the FSA or CMF file is imported, the software will create the visual image.

Another aspect of the invention concerns a feed back of measurement data which is not acceptable to the vendor lab. This can result in a continuous control and feedback after each executed step at the workstation, which automatically or semi-automatically leads to a re-start of the procedure, controlled by the process controller and which is configured by the data template.

### BRIEF DESCRIPTION OF THE DRAWINGS

FIG. 1 shows a bock diagram of a portion of the system.

FIG. 2 shows an illustrative overview of the present method.

FIG. 3 a block diagram of the structure of the system.

FIG. 4 shows a schematic of a template.

FIG. 5 shows a schematic drawing of the system for automated forensic screen.

FIG. 6 shows a schematic drawing for an automated forensic screening method.

FIG. 7 shows an allelic ladder.

FIG. 8 shows positive controls.

FIG. 9 shows negative controls.

FIG. 10 shows a profile for a sample.

FIG. 11 shows standard curve for QUANTIFILER for Example 1.

### DETAILED DESCRIPTION OF THE PREFERRED EMBODIMENTS

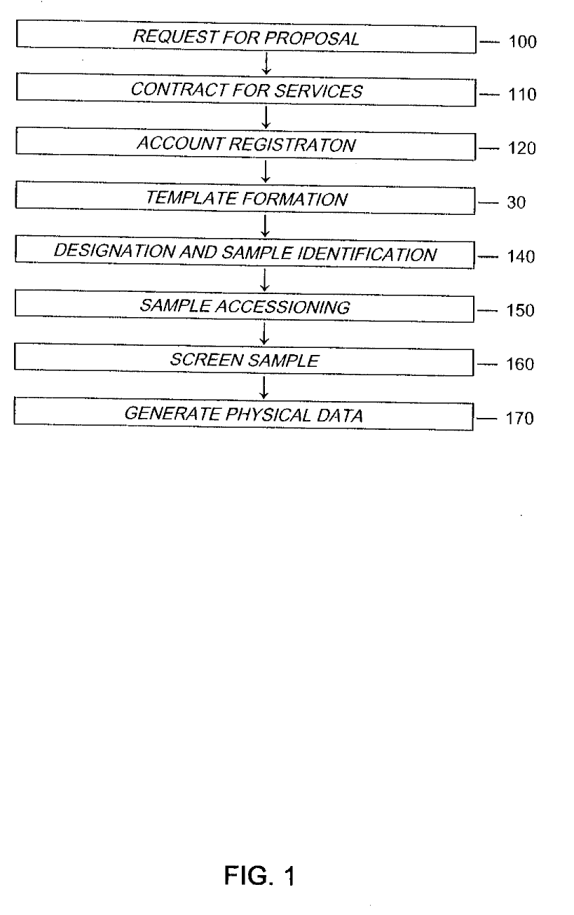

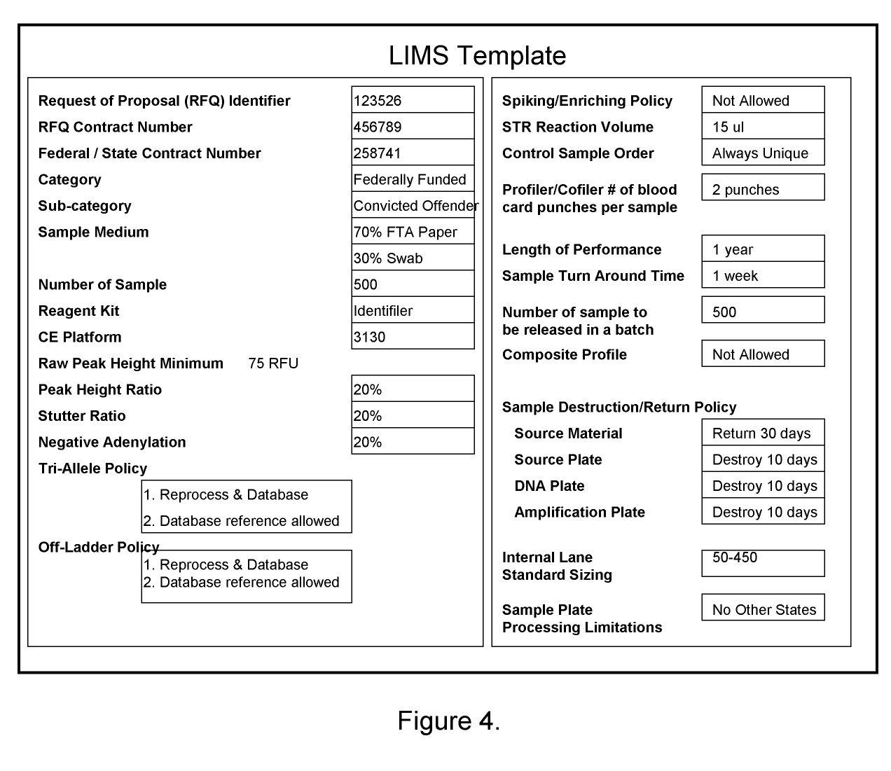

Now referring to FIGS. 1-3, a general overview is shown. A crime investigation entity such as a state crime laboratory, Federal Bureau of Investigation or a law firm representing a person accused of a crime, i.e. a user 1 can publish a request for proposal 100, or in the alternative can directly request services from the vendor laboratory 20. Upon review of the responses, a contract for services 110 with a vendor laboratory 20 is executed with the user 1. After the request for proposal 100, and a contract for services is executed 110, the vendor laboratory 20 website 19 will transmit an access enabling response to the user 1 via electronic communication link 7. This response includes three distinct sections. The three sections are Account Registration 120, Template Formation 30 and Sample Identification and Designation 140. An example of a template 30 is shown as FIG. 4. The template 30 is a database in the laboratory management system LIMS 24 of the vendor laboratory 20.

In the next step of the process a physical sample 40 in a package is sent to the vendor laboratory 20. The vendor laboratory 20 accessions the sample 150, screens the sample 160 and generates physical data 170. The sample 40 has a computer readable identifier that is used to maintain the chain of custody through the process. The physical data 70 is stored in LIMS 24.

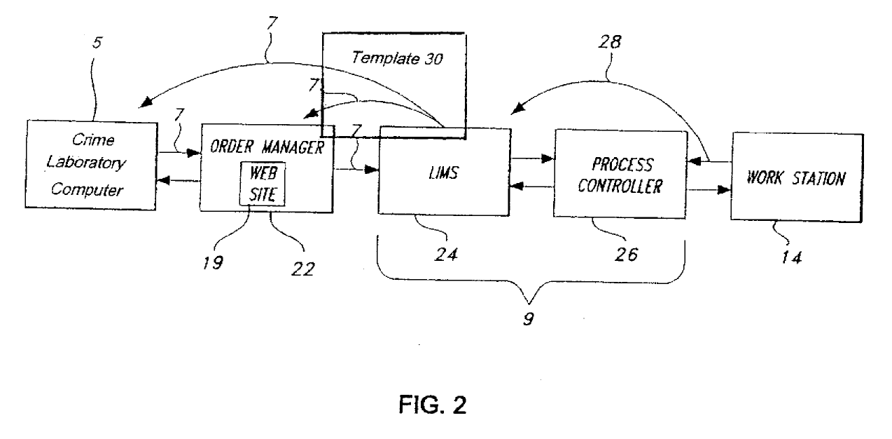

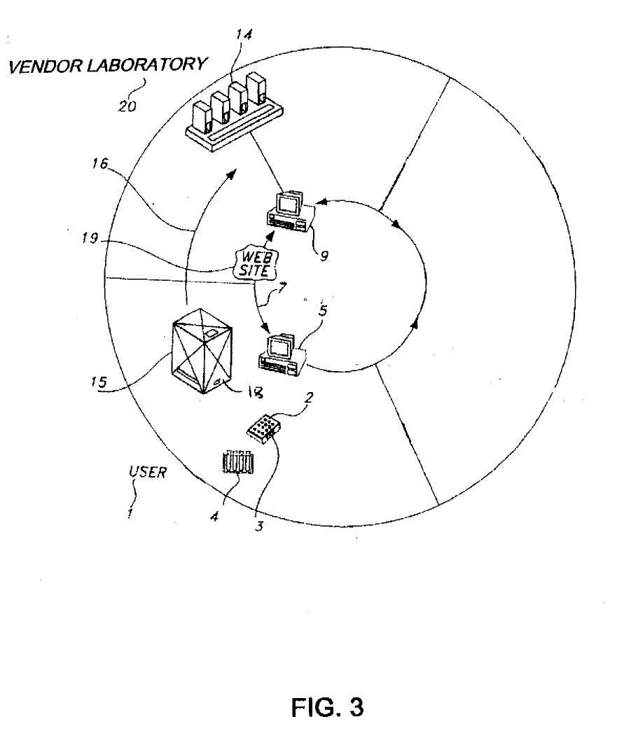

Now referring to FIGS. 2-3, a user 1 provides data which is required for generating a template 30 to a vendor laboratory 20. The vendor laboratory 20 populates the laboratory information management system “LIMS” 24 with the criteria for each user 1. The present invention allows a user 1 with access to a computer 5 to order screening of sample they submit to vendor laboratory 20. Using the secured Internet or other communication link 7, the user 1 sends an access request from the crime laboratories computer 5 to a vendor laboratory 20 computer 9; via an electronic communication link 7, such as the Internet. A user 1 can access vendor laboratory 20 website 19 via communication link. The website 19 can be housed by an Order Manager 22. An Order Manager 22 is a software-based order management system. The Order Manager 22 functions to manage the placement of the order of sample. The order received from the user 1 is transmitted to website 19, which reports the order to Order Manager 22. The Order Manager 22 provides electronic communication with the vendor laboratory 20 computer 9. The vendor laboratory 20 computer 9 includes LIMS 24, which can be communicatively coupled to a process controller 26.

LIMS 24 is the generic name for laboratory LIMS 24 software. The function of LIMS 24 is to be a repository for data, control automation of a laboratory, track sample, chart work flow, and provide electronic data capture. LIMS 24 can also be in direct communication with the user 1 via an electronic communications link 7. Any standard laboratory information system software can be used to provide these functions. The process controller 26 is communicatively coupled to the workstation 14. The process controller 26 provides commands to any portions of the workstation 14 that are amenable to automation. The workstation 14 is communicatively linked to LIMS 24. In this way, the workstation 14 can provide data to LIMS 24, and then, via link to the Order Manager 22 or user 1. In an alternative embodiment, the user 1 computer 5 can be linked to the vendor laboratory 20 computer 9 by a direct phone line, cable or satellite connection.

Now referring to FIG. 4 a sample 40 template is show. Based on the information provided by the user 1, a LIMS 24 Template 30 is created in the vendor laboratory LIMS 24. The information that is captured in LIMS Template 30 includes the state or federal contract number. Additionally, the type(s) of sample 40 is also recorded (i.e. All Arrestee, Convicted Offender, Paternity or Casework). The sample medium and percentages of different sample 40 mediums are also input into the LIMS Template 30. The sample medium can include liquid blood, swabs collected directly, swabs utilized as a transport vehicle which is seen with touch or crime scene evidence, blood cards, clothing, bedding, biological tissue, paper or any other medium that has biological matter. A blood card is any paper type medium which blood is spotted on and dried. The LIMS Template 30 will also include the number of sample that will be submitted under this particular contract. During the accessioning process, LIMS 24 records the user's 1 unique identifier, such as barcode numbers that are affixed to the outside packaging of the sample 40 received from the user 1.

The user 1 will dictate the autosomal (including high-resolution autosomal kits for degraded DNA or low copy number such as APPLIED BIOSYSTEM (Foster City, Calif.) MINIFILER kit), mitochondrial or Y chromosome STR kit type that is to be used on these particular sample and recorded in LIMS 24. The electrophoresis platform such as the 3100 series will be captured in the LIMS Template 30. There are a number of thresholds that are also characterized in the LIMS Template 30. These thresholds include the raw peak height, the peak height ratio cut off, the stutter ratio cut off, the negative adenylation cut off (−A) and the percent shoulder cut off. Other technical parameters that are detailed in the LIMS Template 30 include how to process tri-alleles and off ladder alleles. LIMS 24 will record the spiking and enriching standard operating procedure of each user 1. The reaction volume and limitations will also be outlined and recorded in LIMS Template 30. For States using PROFILER/COFILER the template 30 will dictate the number of sample to be punched per profile for blood cards. The template 30 will determine the length of performance and be recorded. The length of performance is the amount of time that the user 1 anticipates sending the contracted amount of sample. LIMS Template 30 will record the turnaround time necessary for a particular contract. Additionally, the template 30 will dictate how many samples 40 are to be released on a specific timeline. The template 30 will speak to the possibility of composite profiling and procedures on sample 40 return and or destruction policies of the original sample 40 as well as the isolated DNA and the amplified DNA. The LIMS Template 30 will also dictate how much of the sample 40 the vendor lab can use when performing screening. The LIMS Template 30 will define the Internal Lane Standard sizing range. Tri-Allele is the detection of a third peak for a particular loci and off-ladder allele are peaks that are deleted outside the bins of the allele ladder or otherwise defined.

In one embodiment, template 30 is populated into LIMS 24 then, user 1 can order forensic screening of samples 40 they submit to vendor laboratory 20. Using the Internet or other communication link 7, the user 1 sends an access request 7 from the remote user's computer 5 to a vendor laboratory 20 computer 9 via an electronic communication link 7, such as the Internet. The screening laboratory website 16 will transmit an access enabling response to the user 1 via an electronic communication link, such as the Internet. In an alternative embodiment, the account registration 120, template formatting 30 and designation sample identifier 140 can occur at the same time.

Now referring to FIG. 3, a user 1 can access vendor laboratory 20's website 19 via a communication link 7. The website 19 can be housed by an order manager 22. An order manager is a software ordering management system. The order manager 22 functions to manage the placement of the order and houses the web site 19. The order received from the user 1 as recorded in the website 19, is reported to order manager 22, which is in electronic communication 7 with the vendor laboratory computer 9. The vendor laboratory 20 computer 9 includes LIMS 24, which is communicatively coupled to a process controller 26.

The process controller 26 is communicatively coupled to the workstation 14. The process controller 26 provides commands to any portions of the workstation 14 that are amenable to automation. In this way, the workstation 14 can provide data to LIMS 24 for the formulation of the forensic report 100, via an electronic communication link 7, such as the Internet, to the order manager 22 or user 1. In an alternative embodiment, the remote user 1 can be linked 7 to the screening laboratory 20 by a direct phone line, cable or satellite connection.

The Account Registration 120 section begins with logging into the system. A user 1 accesses an existing account by entering an account identification, which is, for example, an e-mail address. The user 1 will then enter a password. If a valid password is entered, the user can place a new order. Alternatively, the user can check an order status by providing an order number or unique contract number and can proceed to order or sample 40 tracking. Alternatively, a new account can be opened by providing an institution name, principal investigator/CODIS administrator etc., contract number, address, phone number, fax number, electronic mail address, billing information, and other authorized user names and unique identifiers. The user 1 can enter a password and confirm the password.

For each account registration a Template 30 will be populated for each unique contract, Project or Order. The Template 30 section contains the information that is recorded as the LIMS Template 30 by the vendor laboratory LIMS 24. A Template 30 section may be populated by the vendor laboratory 20 based upon the specific requirements of the user 1. The template 30 section may or may not be configurable or editable by the user 1 via the Web.

Once the user 1 has the Template 30 section populated, the user 1 will be presented with the Contract Designation Section & Sample Identification 140. In Contract Designation Section & Sample Identification 140, the user identifies which Template 30 (LIMS template) should be applied to the samples 40. The LIMS Template 30 may be identified and selected by the unique contract number by the user 1.

Further the user 1 identifies where each sample 40 to be tested is located in an actual (physical) container by associating each sample 40 with a corresponding well of a virtual 96 well container displayed on the computer screen of computer as described below. The Designation Section & Sample Identification 140 section includes 96 well container locations. The user 1 designates which sample 40 was or will be placed into each well. This designation may occur at the user 1 computer or vendor laboratory computer depending on how the vendor receives the samples. The designation occurs using a barcode wedge or scanner on the unique barcode from the crime laboratory l's LIMS 24 that is affixed to each sample 40 packaging. Alternatively, if the barcode is unreadable using a barcode wedge the information may be manually populated using a keyboard. If the user 1 has more than 96 samples 40, subsequent 96 containers and designations are available. A 96 well container having a barcode accession number is shown oriented in the longitudinal direction having an X axis labeled “A” to “H” and a Y axis labeled “1” to “12”. The X and Y axes designate a well position such as “A1”. Additionally, the genetic sequence to be screened is identified in a computer readable format.

The user 1 or vendor laboratory 20 is asked to provide the container accession number. The user 1 gets the accession number from the physical source micro well container 2 or tube rack 4 containing barcoded tube, that they intend to fill (or has filled) with the samples 40. Additionally, the user 1 is asked to provide the number of contracts represented with the submission, number of samples 40, and sample 40 identification number for a particular well location.

At this point, the user 1 has completed the Template 30 section and the Designation Section 140 and is ready to transmit the screening parameter selections gathered in those sections to website and thence to vendor laboratory 20 computer. The transportation package identifier, sample identifier and at least one genetic sequence is stored in the LIMS 24 in association with the template 30.

The user 1 transmits his or her order to the vendor laboratory 20 via a link such as the Internet or a direct line. The user 1 can transmit the selected data to LIMS 24 in the vendor laboratory 20 via electronic communications link 7. This link can be direct or indirect. In the indirect route, the screening parameters are first transmitted to website, wherein Order Manager 22 receives the order and then provides LIMS 24 with the screening parameter selections.

Once all groups of samples 40 have been entered and listed in the table on the revised first ordering webpage, the operator then selects a button identified “next” and moves to the next stage in the ordering process. Computer transmits this request to website, which generates a graphical image of a 96 container, appearing on the screen of computer identical to the corresponding 96 container that the user 1 or vendor laboratory 20 is filling/has filled with samples 40, and transmits that image embedded in a second webpage back to computer for display. The second webpage includes a graphical representation of a 96 well plate, in a top view, showing the two dimensional array of all 96 wells in which the user 1 is to place the samples 40 identified previously. Website calculates the respective positions of each group of samples 40 in the well container. Each group is shown in the graphical representation of the well plate in a different color. The images of the wells in the webpage are displayed on the computer with an initial shading to indicate that they have not been identified to a particular sample 40. In the preferred embodiment, each well contains a sample 40, such as a blood card, swab or swab cutting, tissue sample 40 or cutting of article of interest taken from an individual or source. The purpose of the testing performed on the samples 40 in the wells is to determine the genetic characteristics (or profile(s)) from which each sample 40 was taken. In order to relate the test results performed on each sample 40 back to the unique individual or unique article of interest from which the sample 40 was taken, the user 1 must make a record of the individual source of each sample 40 (i.e. the barcode number from the crime laboratory LIMS corresponding to the sample 40 or article of interest).

To uniquely identify each sample 40 in each well with an associated sample 40, the user 1 selects a button on the third ordering webpage. This button signals computer to generate an additional webpage. This webpage lists each well in the well plate that was previously identified as containing a sample 40. Each entry in the column of entries includes a well identifier (called well location, above), which is a string of alphanumeric characters that uniquely identifies one well of container. A preferred well identifier for the 96 well plate is an alphabetic character followed by a numeric character. A text box is adjacent to each well identifier on the additional webpage. To uniquely identify each sample 40 in the container, the user 1 enters alphanumeric characters in the text box that are uniquely associated with each sample 40. This identifier is typically a short string of consecutive alphabet or numeric characters, a practice commonly used by crime laboratories to identify individuals and is a computer readable format.

To assist the user 1 in entering the sample 40 identifications into each of the text boxes in the additional webpage, a button is provided to automatically fill several consecutive text boxes based upon the alphanumeric characters typed into a few text boxes from the group. For example, if the user types in “B1234” in the first text box of a group, then types in “B1235” in the second text box of a group, computer is configured to automatically generate consecutive alphanumeric strings to fill the remaining text boxes of the group based upon these two manually typed-in entries. In this case, computer would automatically generate the alphanumeric strings “B1236”, “B1237”, “B1238”, etc. and insert these characters sequentially into the remaining text boxes of the group in the additional webpage. This process can be repeated for each subsequent group shown on the additional webpage. Alternatively, the computer can be configured to automatically generate alphanumeric characters for all the groups at once and to fill the text boxes of all the groups all at once. The automatic fill functionality is built to provide convenience for the user 1 however, some crime laboratories may choose to not utilize this functionality because the standard operating procedure and/or chain-of-custody concerns.

Once the operator has entered the tracking number and the expected shipping date, he clicks on a button labeled “confirm order”, which transmits the completed order, including the tracking number and expected shipping date to website and Order Manager 22, and thence to LIMS 24.

In the preferred embodiment, the STR reagents are placed in workstation 14 and LIMS 24 will record the barcode of the reagents and record their specific location on the deck of the workstation 14, as will be discussed in more detail with respect to the Amplification Plate Building Station 64. Additionally, the vendor laboratory 20's LIMS 24 correlates which reagents or STR kits will be used on which samples 40, as will be discussed in more detail with regard to the Amplification Plate Building Station 64.

As can be seen from FIG. 3, a biological sample 40 is identified with a sample 40 identifier by the user 1. The sample identifier can also include a well plate accestion number 3 or a test tube rack number 4. The biological sample 40 is transmitted from the user 1 to the vendor laboratory 20 in a package 19 with a package identifier 18. The crime laboratory 1 places the appropriate samples 40 into the containers previously identified in the order sent to website 19, Order Manager 22 and LIMS 24. In other words, the user 1 fills each well of container such that each well contains the same sample 40 with the same sample identification that the user 1 previously identified in the order previously sent to website 19.

The container can be a 96 well plate 2, individual 2-D barcoded tubes 4, or the like that receives the sample 40 in each well of the well plate. A sufficient amount of lysis reagent can be added to cover the sample 40 or a portion of the sample 40. In one embodiment, the lysis reagent is added prior to transit to the vendor laboratory 20. Although, in the preferred embodiment the lysis reagent is added at the vendor laboratory 20 at a Lysing Station 54.

In the preferred embodiment, the containers are populated at the vendor laboratory 20. The overnight carriers tracking number of the user 1 shipping label is read with a barcode reader. If the shipping label is unreadable, the tracking numbers are manually entered. The scanning of the tracking number is received in LIMS 24 and a received message is electronically generated to the point of contact inside the user 1 following accessioning. The container are populated with the accessioning techniques at the vendor laboratory 20, as described above, and taken to a clean room. The containers contain the raw biological matter and in one embodiment lysis reagent. The containers individual barcodes and/or individual to barcodes are scanned by the barcode reader and recorded in LIMS 24 as accession numbers. If the containers individual barcodes are unable to be scanned, the accession numbers are entered manually. If the overnight carriers tracking number, accession number, user order and worklist properly correlate, LIMS 24 will activate an active record number for the containers.

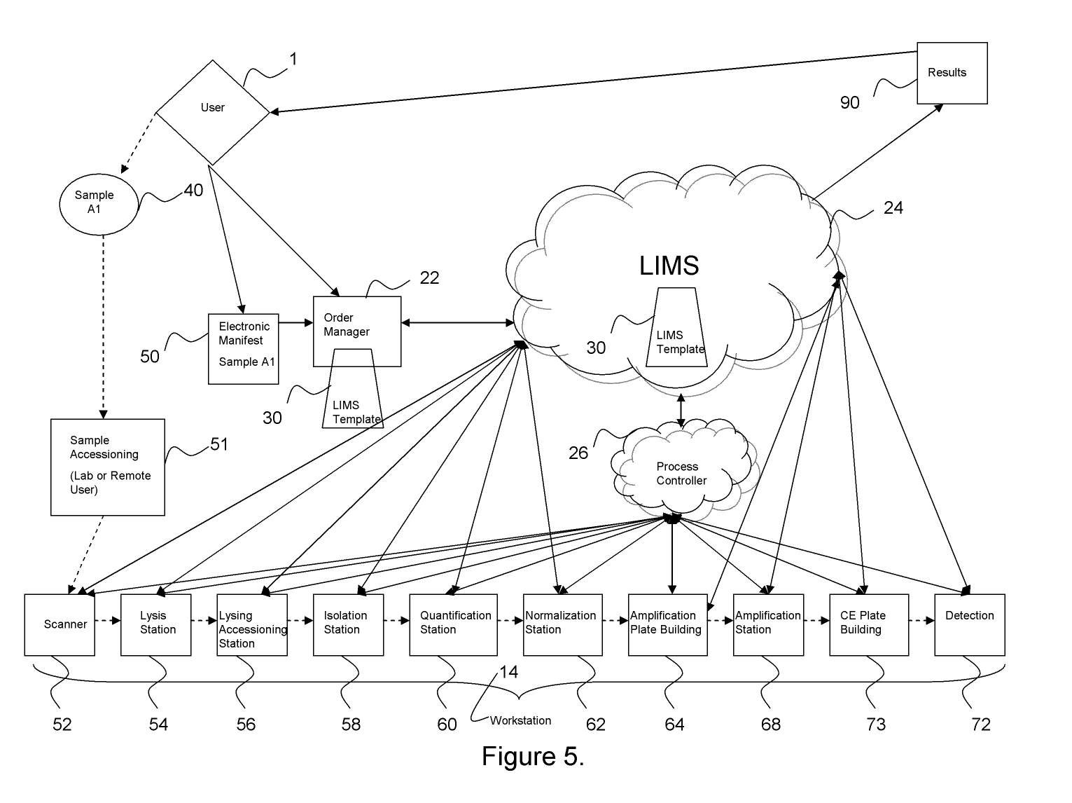

Now referring to FIG. 5, the sample 40 is transmitted to the vendor laboratory 20. The shipping container is sealed and the vendor laboratories barcode 18 shipping label is affixed to the outside of the package 15. The overnight shipping company transports the samples 40 to be screened to the vendor laboratory 20. The vendor laboratory 20 receives shipment via the overnight courier from the user 1. The vendor laboratory 20 scans 52 the overnight carrier's barcode 18 in a receiving area. LIMS 24 automatically generates an e-mail to the user 1 point of contact confirming that the shipment was received intact, if it was so

The vendor laboratory 20 takes receipt of the crime laboratories electronic shipping manifest 50 via any electronic media. This may be an electronic communication as simple as a disk received in the shipping container with the samples 40. Alternatively, the electronic manifest 50 may be received by the vendor laboratory 20 via an FTP site, e-mail or any other electronic communication. The vendor laboratory 20 may also take receipt of a physically printed shipping manifest if electronic media is not available. Operators at the vendor laboratory 20 will convert this physically printed manifest into an electronic version 50 via a computer configured to record such information in the vendor laboratories LIMS 24.

The samples 40 received by the vendor laboratory 20 that have the chain of custody intact will be accessioned 51 either remotely if the sample 40 is in a well plate or is in test tube or manually if the samples 40 is not in a test tube or well plate. If manual accessioning is required the samples 40 are sorted into bins of different sample 40 type. In the accessioning area 51 of the vendor laboratory 20, an operator would enter the respective contract number into the accessioning computers. This contract number would be used to affiliate LIMS Template 30 with the samples 40 received from the user 1 and further affiliated with the barcoded (or RFID) tubes (or other consumables such as a microwell plate) of the vendor laboratory 20. The vendor laboratory 20 barcodes or RFID unique identifiers will link the unique crime lab samples 40 to specific consumables used by the vendor laboratory 20 during processing.

Once the accessioning 51 has been configured by the vendor laboratory operator, the accessioning of the different types of bins may proceed. Swabs are typically accessioned using two different manners; automated and manual. The first automated technique utilizes a laser cutter while the second technique is a manual based cutting system.

A laser cutter device, such as those sold by BSD ROBOTICS (Queensland, Australia) is a fully automated or semi-automated device used to cut swabs and place them into unique locations of microwell plates. In the preferred embodiment of the system a barcode tube rack, like that from MICRONICS (McMurray, Pa.), is scanned and recorded into the LIMS 24. Empty 2-D barcoded tubes are placed in this tube rack. The scanning of the empty 2-D barcodes is done from the bottom of the tube rack once all of the empty 2-D barcode tubes are loaded into the rack. This scanning confers a specific location for each tube within that rack. The tube rack and tubes are loaded into the laser cutter device. The barcode on the outside of the individual sample 40 packages from the user 1 is scanned via a barcode wedge. If the barcode is not readable using the barcode wedge the barcode number can be keyed in manually and is recorded into LIMS 24. As each of these unique barcodes is scanned with a barcode wedge, LIMS 24 deselects the samples 40 from the electronic shipping manifest. This is the electronic shipping manifest that was sent by the user 1 to the vendor laboratory 20 and uploaded into the vendor laboratories LIMS 24. The package 15 is then opened to allow access to the sample 40. Computer entry is then made of the sample type and recorded into the LIMS 24. The LIMS 24 also records the number of samples 40 that were originally received in the package. Computer entry is made to note insufficient sample 40 or damage samples 40. At the end of the entire accessioning process and e-mail is be generated to the point of contact of the user 1 to indicate to them the samples 40 issues. The samples 40 that are deemed to be sufficient continued the process. Vendor laboratory 20 barcodes are printed and affixed to the swab and the original packaging. One barcode is placed in a predefined location on the original packaging. The original package 15, which may have additional samples 40, is then resealed and stored according to the vendor laboratory 20 sequential barcode number.

The individually barcoded swab sample 40 may or may not be placed into a secondary holder (a device that holds the handle portion of the swab), which itself is barcoded, to allow proper seating into the laser cutter device. The swab is placed into holding position, or device such as a magazine, in the laser cutting device. Sample 40 is then opened and accessioned as described above until all samples 40 have been accessioned or the capacity of the laser cutting device or the capacity of the 2-D barcoded tubes or micro well plate(s) is exceeded. If the holding mechanism or magazine is populated with samples 40 outside of the laser cutting device then the holding mechanism will be moved inside the laser cutting instrument. The cutting program is then executed to scan the barcodes on samples 40 and to cut each sample 40. The cutting program has the ability to create files which are important for the chain of custody. These files may be created as ASCII, XML, CSV, Text or any other file format containing the test name, the plate number, the plate barcode, the cell reference, the cell barcode, the spot type, the fill order, cell alias or any other comments. The electronic file information generated by the swab cutting program is recorded into LIMS 24. The rack containing the sample 40 filled 2-D barcode tubes is then removed from the laser cutter device. The 2-D barcode tubes containing the samples 40 are then capped. The samples 40 still residing in the magazine or swab holder are then removed sequentially. Each cut sample 40 has its barcode scanned with a barcode wedge and the original crime laboratory sample 40 packaging, bearing the vendor laboratory 20 barcode, has its barcode scanned. If the two barcodes match as indicated by the accessioning computer, the cut sample 40 is returned to the original packaging.

Blood cards can also be accessioned using a fully automated process, a semi-automated process or a manual process. Blood cards are meant to include samples 40 received by the vendor laboratory 20 in which biological sample 40 such as whole blood is applied directly to the substrate. Typically, these substrates or cards include paper (Schleicher & Schuell) and FTA PAPER (Whatman), with or without an indicator dye. However blood cards may include any other substrate such as any other cellulose paper.

Semi-automated blood card accessioning begins with the scanning of the user 1 barcode on the outside of the individual sample 40 packages using a barcode wedge. If the barcode is not readable using the barcode wedge the barcode number can be keyed in manually and is recorded into LIMS 24. As each of these unique barcodes is scanned with a barcode wedge, LIMS 24 deselects the samples 40 from the electronic shipping manifest. This is the electronic shipping manifest is typically sent by the user 1 to the vendor laboratory 20 and uploaded into the vendor laboratories LIMS 24. A barcode tube rack, such as the Micronics tube rack, is scanned and recorded into LIMS 24. Empty 2-D barcode tubes are placed into the tube rack and have their barcode scanned from the bottom. This scanning correlates a unique tube identifier to a unique position in the tube rack. The tube rack containing the 2-D barcoded tubes are loaded into the semi-automated punching machine. The user 1 sample packaging 15 is then opened and computer entry is made. LIMS 24 records the sample 40 type within the packaging 15. Computer entry and recording is made of the number of samples 40 in the packaging 15. A computer in the accessioning area 51 is configured to have the ability to record, insufficient samples 40, missing samples 40 or damaged samples 40. After all samples 40 have been accessioned 51 an automated e-mail is generated to the point of contact at the user 1 disclosing the samples 40 that had issues. Individual blood card samples 40 may have unique identifying markers or barcode numbers on the samples 40 that may be recorded into the LIMS 24. The semi-automated card punching is then executed. The barcode of the blood card is scanned using the semi-automated punching device. The punching is then initiated. Typically, punching is done by holding an individual blood card in a specific position and engaging a trigger mechanism, such as a foot pedal. Many times a single punch is all that is needed to generate a DNA profile. However, there are instances where two punches are required to generate a profile. Such is the case with PROFILER PLUS and COFILER for some state crime laboratories.

A computer in the accessioning area 51 is configured based on the LIMS 24 Template 30 to not allow a different barcode to be punched if a sample 40 requiring two punches is not complete. The semi-automated punch device creates a unique file such as an ASCII, XML, CSV, Text or any other file format. These files may containing the test name, the plate number, the plate barcode, the cell reference, the cell barcode, the spot type, the fill order, cell alias or any other comments. Information from this file is recorded into LIMS 24. The next step requires the blood card samples 40 barcode to be scanned and the sample 40 packaging barcode to be scanned. If the two barcodes match the blood card is returned to the original packaging. However, if the barcodes do not match accessioning computer is configured to warn the operator. The package now containing the punched sample 40 and any additional replicate samples 40 and is resealed and stored sequentially according to the vendor laboratory 20's barcode number and chain of custody procedure. The next crime laboratories sample 40 package is then accessioned. Sample Accessioning area 51 continues in this manner until the tube rack reaches capacity or all of the samples 40 for this contract have been accessioned. The barcoded tube rack and the 2-D bar coded tubes, which now contain the samples 40, are removed from the semi-automated punch. The tubes in the rack are then capped.

Other evidence from a crime scene, such as clothing materials may also be processed in an automated, semi-automated or manual procedure following standard presumptive or confirmatory test. The manual accessioning and processing of clothing type materials begins with the scanning of the barcode on the outside of the sample 40 package from the user 1. This unique user 1 barcode identifier is recorded into the vendor laboratory 2 LIMS 24. The barcode on the outside of the individual sample 40 package is typically recorded using a barcode wedge. If the barcode is not readable using the barcode wedge the barcode number can be keyed in manually and is recorded into LIMS 24. The vendor laboratory LIMS 24 deselect the sample 40 numbers off of the electronic shipping manifest that was sent by the user 1 and received by the vendor laboratory 20. The accessioning computer is configured such that computer entry is made of the sample 40 type and may have a description field. Computer entry is made of the number of samples 40 inside the package. The accessioning computer is configured to allow the operator to comment missing or damage samples 40. After all samples 40 have been accessioned an electronic communication such as an e-mail is generated to the point to contact at the user 1 to disclose the issues with the relevant samples 40.

In certain instances manual cuttings cannot be performed until the area of interest is identified on the article of clothing. The sample 40 location(s) may be identified through the use of different techniques such as alternative light source. Once an area of interest is identified a manual cutting around the biological material is performed.

Presumptive or confirmatory test as part of accessioning area 51 are routinely used on the biological matter or a portion of the biological material found on the article of interest. Clothing materials or portions of clothing material may undergo a variety of different treatments prior to being accessioned. These treatments may include confirmatory tests such as the direct observation of sperm. This is done by applying recovered sperm cells onto a microscope slide and heat fixed. Immobilized cells are stained with a combination of stains, such as Christmas Tree stain, and visualized under a light microscope.

Chemical treatments may include presumptive test to identify if the sample 40 has a particular biological source. Commonly these confirmatory or presumptive tests are used to determine if a biological sample 40 is semen and or seminal fluid, vaginal secretion, human blood, or animal blood. Additionally, presumptive test may include saliva, urine or feces. Many of these common biological materials of forensic interest can be detected with standard serological testing.

Convenient commercial products may also be used for presumptive and confirmatory testing. Seminal fluid may be detected by THE SERATED PSA SEMIQUANT (SERATEC DIAGNOSTICA, Göttingen, Germany) membrane, and the ONESTEP ABA CARD P30 (Abacus Diagnostics, West Hills, Calif.). These detection methodologies utilize immunochromatographic membranes that allow for rapid, convenient, and highly sensitive analysis for the presence prostate specific antigen (PSA) or also known as P30.

A simple and convenient commercially available human blood detection kit is available from abacus diagnostics (West Hills, Calif.) as the ABA CARD HEMATRACE kit. The kit is based upon the detection of human hemoglobin. Another presumptive test for human blood is luminol. A presumptive test for amylase is used to indicate the presence of saliva. Two common methods for estimating amylase levels in forensic samples 40 include the Phadebas test and the starch iodine radial diffusion test.

Following confirmatory or presumptive testing sample 40 cuttings are made of the area(s) of interest from the article of clothing. Sample 40 cuttings may be performed with a variety of instruments including scalpels, scissors, automated or manual punches or any other mechanical device to excise the biological material from the article of interest. In certain instances it is possible to transfer biological material to an absorbent carrier such as a swab that can be processed in multiple fashions as described above. Biological material can be transferred from an article of interest to a swab by a wetting the tip of a sterile swab with the deionized water and running at over a particular material to collect biological material of interest.

Once the cuttings are collected from the clothing or article of interest they are placed into 2-D barcode tube. This 2-D barcode tube is then read the single tube barcode scanner. LIMS 24 affiliates this barcode number with the vendor laboratory 20 barcode number. The vendor laboratory 20 computer is configured to print a barcode that has the same unique identifier as the 2-D barcode tube and is affixed to a predefined location on the original user 1 packaging. The original article of interest is returned to the crime lab original packaging. The packaging is resealed and stored according to the sequential vendor laboratory 20 barcode number. The 2-D barcode tube containing the sample 40 is placed into a bar coded tube rack. The next sample 40 may be opened and accessioned as described above. Once all the samples 40 are accessioned or the tube rack reaches capacity, then the 2-D bar coded tubes are scanned from the bottom while in the barcode tube rack. This scanning correlates a unique barcode identifier to a unique location inside the bar coded tube rack and is recorded in LIMS 24. The tubes are then capped for further processing.

Semi-automated, clothing or article of interest, accessioning begins with the scanning of the user 1 barcode on the outside of the individual sample 40 packages using a barcode wedge. If the barcode is not readable using the barcode wedge the barcode number can be keyed in manually and is recorded into LIMS 24. As each of these unique barcodes is scanned with a barcode wedge, LIMS 24 deselects the samples 40 from the electronic shipping manifest. This is the electronic shipping manifest that was sent by the user 1 to the vendor laboratory 20 and uploaded into the vendor laboratories LIMS 24. A barcoded tube rack, such as the MICRONICS tube rack, is scanned and recorded into LIMS 24. Empty 2-D barcode tubes are placed into the tube rack and have their barcode scanned from the bottom. This scanning correlates a unique tube identifier to a unique position in the tube rack. The tube rack containing the 2-D barcoded tubes are loaded into the semi-automated punching machine. The crime lab sample 40 packaging is then opened and computer entry is made. LIMS 24 records the sample 40 type within the packaging 15. Computer entry and recording is made of the number of samples 40 in the packaging. The accessioning computer is configured to have the ability to record, insufficient samples 40, missing samples 40 or damaged samples 40. After all samples 40 have been accessioned an electronic communication such as an automated e-mail is generated to the point of contact at the user 1 disclosing the samples 40 that had issues. However, since not all states crime laboratories have the same standard operating procedure the vendor laboratory 20 will execute a barcode procedure that prints two identical barcodes. One of these barcodes will be affixed to the article of interest while the other barcode will be affixed to the packaging 15 in a predefined location. The barcode of the clothing or article of interest is scanned at the semi-automated punching device. The punching is then initiated. Typically, punching is done by holding an individual item in a specific position and engaging a trigger mechanism, such as a foot pedal. The semi-automated punch device creates a unique file such as an ASCII, XML, CSV, Text or any other type of usable file. These files may contain the test name, the plate number, the plate barcode, the cell reference, the cell barcode, the spot type, the fill order, cell alias or any other comments. Information from this file is recorded into LIMS 24. The next step requires the clothing samples 40 barcode to be scanned and the sample 40 packaging barcode to be scanned. If the two barcodes matched the clothing or article of interest is returned to the original packaging. However, if the barcodes do not match accessioning computer is configured to warn the operator. The package now containing the punched sample 40 and additional samples 40 and is resealed and stored sequentially is according to the vendor laboratory 20's barcode number. The next crime laboratories sample 40 package is then accessioned. Accessioning continues in this manner until the tube rack reaches capacity or all of the samples 40 for this contract have been accessioned. The barcoded tube rack and the 2-D bar coded tubes which now contain the samples 40 are removed from the semi-automated punch. The tubes in the rack are then capped.

In this system, the operator at the vendor laboratory 20 loads containers into the transportation apparatuses after the containers are received at the vendor laboratory 20 for web-based orders. Alternatively the vendor laboratory 20 loads containers into transportation apparatuses after the containers are created utilizing the above described vendor laboratory 20 accessioning process. Irrespective of the methodology of accessioning the vendor laboratory 20 scans 52 into LIMS 24 the containers unique identification as described above for transportation to the workstation 14. Alternatively, containers can be transported individually to workstation 14 and be placed in transportation apparatuses that are already located at workstation 14.

An automated laboratory work station is a logical groupings of laboratory operations. These groupings, however, do not necessarily refer to different physical stations. These logical groupings include: Lysing Station 54, Automated Lysate Accessioning Station 56, Isolation/Purification Station 58, Sample Normalization Station 62, Real-Time PCR Quantification Station 60, Amplification Plate Building Station 64, Amplification Station 68, Capillary Electrophoresis Plate Building Station 73 and Detection Station 72, all of whom make up the workstations.

The process controller 26 is communicatively coupled to LIMS 24. The process controller 26 information received from LIMS 24, in the form a worklist or corrective action worklist, varies depending on the type of instrumentation. For example, the Lysing Station 54, Automated Lysate Accessioning Station 56, Isolation/Purification Station 58, Sample Normalization Station 62, Real-Time PCR Quantification Station 60, Amplification Plate Building Station 64 and Capillary Electrophoresis Plate Building Station 73 are liquid handlers/pipetting devices. The process controller 26 instructs these instruments the volume of reagent or sample to aspirate and dispense, the position from where to aspirate and the position were to dispense. Additionally, the process controller 26 instructs the instrument the height above the instrument deck (Z height) to aspirate and dispense. Further the process controller 26 directs the liquid class that is to be used for each aspirate and dispense. A liquid class is a pippeting technique that can be changed based on the viscosity of the liquid to be aspirated and dispensed.

The instrumentation, such as the Real-Time PCR Quantification Station 60 and Amplification Station 68 also is directed by process controllers 26. LIMS 24 sends the process controller 26 the sample setup and detections parameters via a worklist or corrective action worklist. The Real-Time PCR Quantification Station 60 and Amplification Station 68 process controller 26 directs the performance of the thermocycler elements of these instruments to meet the detection parameters. The process controller 26 directs the thermocyclers' hold times, the hold temperatures, time of each cycle, the temperature of each cycle/step and the number of cycles. Further in the case of the Real-Time PCR Quantification Station 60 the process controller 26 directs the specific detector that is to be applied to each well. The detector is an element that allows a particular fluorescence to de detected and quantified.

The instrumentation, such as the Detection Station 72 also is directed by the process controller 26. LIMS 24 sends the process controller 26 the sample setup and detections parameters via a worklist or corrective action worklist. The Detection Stations' process controller 26 instructs the capillary electrophoresis instrument the amount of time to inject the samples into the capillaries. Further the process controller 26 instructs the capillary electrophoresis instrument the amount of voltage that is to be applied to the samples for injection.

Lysing Station 54 is an alternative step provided to lyse the samples 40 in containers or tubes in the event user 1 does not choose to lyse the samples 40 by adding a lysis reagent before sending them to the vendor laboratory 20 or if the samples 40 are accessioned at the vendor laboratory 20. The functions of the various logical stations are described below. The following description provides the preferred embodiment, although one skilled in the art could elect to conduct these methods with varying degrees of automation as required.

As mentioned above, the user 1 need not add a lysis reagent to the samples 40 before shipping them to vendor laboratory 20. Instead, the samples 40 may be shipped from the user 1 to the vendor laboratory 20 un-lysed (at room temperature, on cold packs or frozen) and may be lysed at the vendor laboratory 20. Lysing is achieved by piercing the cover of a multi-well container or the caps of 2-D barcode tubes and treating each of the samples 40 with a lysis reagent after docking the tray in the workstation 14 in the Lysing Station 54. The samples 40 are incubated to produce a lysate containing cellular debris including nucleic acid.

With respect to the swab and blood cards sample, a sufficient amount of a lysis reagent, purchased from a commercial vendor or compounded by the vendor laboratory 20, is added to each well of containers to cover at least a portion of the sample 40. 100 mL of such a buffer can be created by compounding 95 mL of sterile deionized water with 1 mL Triton, 1 mL 5M NaCl, 2 mL of 0.5M EDTA and 1 mL of IM Tris-HCl (at a pH of 7.5) and 5.172 mL of 10 mg per milliliter proteinase K. The samples 40 are incubated for one hour at 55° C. followed by a 30 minute 95° C. incubation.

Differential extraction refers to the process by which the DNA from two different types of cells can be extracted without mixing their contents. The most common application of this method is the extraction of DNA from vaginal epithelial cells and sperm cells from sexual assault cases in order to determine the DNA profiles of the victim and the perpetrator. Differential extraction occurs by adding a first lysis reagent to a first portion of the sample 40 associated with a sample identifier and a second lysis reagent to a second portion of the sample 40 associated with said sample identifier

With respect to the differential lysis and extractions, a sufficient amount of a lysis reagent, such as phosphate buffered saline is added to each well of containers to cover the sample 40. The samples 40 incubated at room temperature for 60 minutes to rehydrate the sample 40. For stains that are one-year or older this rehydration step may be extended at 4° C. The samples 40 are then vortexed to facilitate the release of any cells from the substrate. Typically, the substrate is a cotton swab such as those found in standard Rape Examination Kits. For sample 40 in which the biological material may have a low yield the substrate may be placed into a Spin Easy extraction basket and spun at 1,000-5,000×g for five minutes in order to recover as much liquid biological materials possible. Alternatively, PROMEGA'S (Madison, Wis.) SLICPREP 96 Device (Cat# V1391) spin basket, or the like, may be utilized to maximize the recovery of liquid containing the biological material. Recovered liquid obtained through centrifugation would then be added to the previously hydrated and recovered portions of the sample 40. The entire extract would be spun for five minutes at 5,000×g. A majority of the liquid supernatant portion would be removed using a liquid handler and placed into the secondary bar-coded multiwell container to be saved and used for presumptive and/or confirmatory biological source testing such as PSA (also known as P30). A small portion of the liquid supernatant (i.e. 50 μL) will remain on the sample 40. The sample 40 and the small (about 50 microliter) supernatant volume is vortexed on the liquid handler.

The liquid handler removes 2.5 microliters of the vortexed epithelial cell solution and applied to an appropriately labeled (bar-coded) glass microscope slide. The slide is then heat fixed for 20 minutes at 55° C. The epithelial cells are stained on the slide by adding 1-2 drops Nuclear Fast Red stain for 15-20 minutes with the automated liquid handler. The slide is washed gently with deionized water. The liquid handler then dispenses one drop of picroindigocarmine stain for 4-5 seconds. The Roma arm of the liquid handler then rinse is the slide with 100% ethanol. The bar-coded epithelial slide is allowed to dry. The slide is then imaged under a light microscope and that images recorded in the LIMS 24. To each 50 μL resuspended pellet in the multiwell container, the liquid handler adds 400 μL of digestion buffer, and then adds 10 μL (20 mg/ml) proteinase K solution. The liquid handler tip mixes, or vortexes, the samples 40 and the multiwell container is incubated at 55° C. (+/−5° C.) for 1.5 hours. The samples 40 residing in the multiwell container are spun in a centrifuge for 5 minutes at 10,000×g. The liquid handler removes the supernatant to another multiwell container that is barcoded and called the “Epithelial fraction”. The Epithelial fraction multiwell container may be stored at −20° C. until time of extraction. The original barcoded multiwell container that contains the pellet is known as the “Sperm fraction”. To the Sperm fraction plate, the liquid handler adds 400 μL of digestion buffer. The Sperm fraction multiwell container is vortexed to resuspend the pellet. The Sperm fraction multiwell container is centrifuged at 10,000×g for five minutes and the automated liquid handler removes the supernatant. This procedure is repeated three times. The automated liquid handler adds 100 μL of sterile deionized water to the samples 40. The sperm fraction multiwell container is vortexed to resuspend the pellet. The sperm fraction multiwell container is centrifuged at 10,000×g for five minutes and the automated liquid handler removes all the supernatant except 50 microliters. The sample 40 and the small 50 microliter supernatant volume is vortexed on the liquid handler.

The liquid handler removes 2.5 microliters of the vortexed sperm cell solution and applied to an appropriately labeled (bar-coded) glass microscope slide. The slide is then heat fixed on the deck of the liquid handler for 20 minutes at 55° C. The epithelial cells are stained on the slide by adding 1-2 drops Nuclear Fast Red stain for 15-20 minutes with the automated liquid handler. The slide is washed gently with deionized water. The liquid handler then dispenses one drop of picroindigocarmine stain and incubates at room temperature for 4-5 seconds. The Roma arm of the liquid handler then rinses the slide with 100% ethanol. The bar-coded epithelial slide is allowed to dry. The slide is then imaged under a light microscope and that images recorded in the LIMS 24. The automated liquid handler then adds 400 microliters of digestion buffer, 20 μL of IM Dithiothreitol (DTT) and 10 μL (20 mg/ml) proteinase K solution to the Sperm fraction multiwell container samples 40. The sperm fraction multiwell container is incubated at 55° C. for 6 to 24 hours.

Each differentially extracted sample 40 may have multiple tests associated with one source sample 40. These tests include the epithelial fraction as well as a sperm fraction that will be independently isolated producing two or more profiles that will be captured and tracked in the LIMS 24. Additionally, other information such as presumptive and confirmatory tests will also be captured in the LIMS 24 for each of the samples 40. All of these individual elements will be linked and tracked by LIMS 24 to a unique barcode which will correlate back to the original sample 40.

The preferred method for performing all the above described lysing steps occurs at the Lysing Station 54. At Lysing Station 54, containers are loaded onto the transportation apparatus and positioned on Lysing Station 54. The Lysing Station 54 includes a liquid handler, such as GENESIS TECAN (Raleigh Durham, N.C.) or MULTIMECK BECKMAN (Indianapolis, Ind.). It includes a frame, on which a deck is mounted to provide a horizontal working surface, which supports a transportation apparatus, which supports and positions up to nine containers. A material handler is fixed to frame and extends upward and across the top surface of deck. A computer is coupled to material handler to direct the movement and operation of pipettes. Troughs or reservoirs are provided on the deck, from which computer commands the material handler to aspirate lysis reagents into pipettes and to deposit the reagent into wells of container or more preferably 2-D barcoded tubes. It should be noted that multiple lysis reagents may reside on the deck at one time. Different lysing agents are used for different types of samples 40. For example the sperm fraction lysis reagents is different than that of the epithelial fraction lysis reagents, which in turn may or may not be different than the standard blood card, swabs/FTA, or swab lysis agent.

The operator first carries a plurality of containers, including tube racks, and places them on the deck of the transportation apparatus in one of the nine positions. The rigid transportation apparatus supports and orients the containers. The transportation apparatus is docked into the workstation 14. The operator manually enters the transportation apparatus unique position number, unique container number and the number of wells that are filled with samples 40 in each of the containers or tube racks into computer. Alternatively, the operator may scan the barcode identifier on the containers, or tube racks, and the bar-coded unique position of the transportation apparatus, which is linked to the accessioning worklist. The worklist is then imported into the material handler computer thus avoiding manual entry by an operator.

Knowing the location of each container, or tube rack, in the transportation apparatus and the number of wells or tubes that are filled with samples 40, the computer then directs material handler to move the pipettes to each container or tube rack in turn piercing the barrier sealing mechanism and filling each of the wells of containers containing a sample 40 with lysis reagent. The barrier sealing mechanism for a container may be a container mat. For 2-D barcoded tubes pre-slit caps serve as the barrier sealing mechanism. By providing the location and the number of samples 40, the computer is configured to fill only the wells/tubes containing samples 40 with the appropriate lysis reagent and volume, leaving the empty wells devoid of lysis reagent.

Once each of the sample 40-containing wells has been filled with lysis reagent, the operator moves the entire transportation apparatus containing the samples 40 to an oven, where the samples 40 may or may not be incubated by heating for a period of about one hour at a temperature of 55° C. depending on the sample 40 type. After the 55° C. incubation the samples 40 may be incubated at 95° C. for 30 minutes. Once the incubation process is complete, the operator moves containers supported on the tray or trays to Automated Lysate Accessioning Station 56.

An Automated Lysate Accessioning Station 56 device removes liquid from the container(s) and moves it to the destination well container. Depending upon the original source material different volumes of lysate may be transferred to the destination well container. The destination well container is the container in which the nucleic acid is isolated. It is preferably a 384 well plate (Fisher Scientific #NC9134044). Any commercially available automated accessioning device can perform this function such as GENESIS TECAN (Raleigh-Durham, N.C.) or MULTIMECK BECKMAN (Indianapolis, Ind.). These devices are referred to as liquid handlers. The containers barcode accession numbers are re-scanned. This measurement will be recorded and posted into the LIMS 24. Additionally, LIMS 24 ensures that containers are consistent from transportation apparatus to the Automated Lysate Accessioning Station 56. The liquid handler utilizes stainless steel, titanium, ceramic, Teflon coated or the like, pipette tips that are washed between each sample 40 transfer within aqueous solutions such as water. Alternatively, the pipette tips may be sanitized with a plasma cleaning device such as the tip chargers sold by Cerionx (Exton, Pa.). Moreover, as in the preferred embodiment, pipette tip cleaning may occur by combination of aqueous washes and plasma exposures. Alternatively, to the fixed tip pipettes, plastic disposable pipette tips may be used.

The nucleic acid lysate is transferred to the clean well containers, called destination well containers. Each of the containers has a unique scannable accession number, preferably a barcode accession number, called “barcodes” or “accession numbers” below. The barcodes of the destination well containers are scanned and LIMS 24 marries the barcodes for the destination well containers to the scanned barcode accession numbers of the source well plates. The automated accessioning of lysate from the containers to the destination well continues until all of the day's pending samples 40 are accessioned into the destination well containers. The transportation apparatus and the containers are taken from the incubating oven back to the same liquid handler that performs the functions of Lysing Station. This liquid handler is also preferably configured to function as Automated Lysate Accessioning Station 56.

The operator returns the transportation apparatus to liquid handler and places transportation apparatus back on the deck of the liquid handler, generally in the same location it was in when the lysis reagent was inserted into each well containing a sample 40.

Once in that location, the operator commands computer to import the work list from LIMS 24 and electronically stores it in the computer memory of process controller 26. The worklist includes the accession numbers of each container that is in tray. The worklist uniquely associates the location of the well, the accession number of container from which the well is from, and all other salient information that was previously recorded in the LIMS Template 30.

Once computer imports the worklist, the computer directs the operator to electronically scan the accession numbers of all the containers that are in rigid tray on deck of liquid handler using scanning device coupled to computer. Scanning device is preferably a glyph scanner, character scanner, bar code scanner, dot matrix scanner, or RFID tag scanner, depending upon the form of the accession identifier (typically a barcode accession number 3) on container. Once containers have been scanned, computer transmits the accession numbers to process controller 26 and thence to LIMS 24. Process controller 26 preferably includes an instrument database to which each of the computers of Lysing Station 54, Automated Lysate Accessioning Station 56, Isolation/Purification Station 58, Sample Normalization Station 62, Amplification Plate Building Station 64, Amplification Station 68, Capillary Electrophoresis Plate Building Station 73 and Detection Station 72 transmit their data in order to maintain an ongoing record of the testing process (chain of custody) and the location of materials and samples 40 throughout that process. Computers of the respective stations then commands material handler to transfer the contents of each well (i.e. lysate) in containers to a corresponding well in the destination well container using pipettes.

Computer directs the operator to scan the accession numbers on the destination well container. Like the accession number on containers, the accession number on the destination well container may be any electronically scannable indicia or device. The computer transmits the accession numbers to process controller 26, which sends them to LIMS 24. In this manner, LIMS 24 maintains a record of each sample 40 and its location in each container and in each destination well container. LIMS 24 and process controller 26 correlate the accession number of each destination well container with the identity of each sample 40 it contains, and the LIMS Template 30 for each sample 40.

The tray of destination well containers is moved to the Isolation Station 58. It should be noted that it is fully contemplated that the Isolation Station 58 may be composed of either vacuum manifold type extraction or spin column/plate isolations. DNA can be isolated using magnetic particles. The amount of DNA isolated from swabs, blood cards, swab/FTA cards and different tissues may differ in DNA yields recovered. The tissue lysate may have enough nucleic acid content to saturate the binding ability of the fixed volume of beads. However, the swab and blood lysate may not have enough DNA to saturate the binding ability of the fixed amount of beads. This is evidence by Real-Time PCR CT (cycle threshold) values for the housekeeping probe. The housekeeping (telomerase probe) CT values for tissue isolations are approximately 25 whereas the approximate CT for housekeeping (telomerase probe) for the blood isolations are approximately 29. This 4 cycle difference represents approximately a 16 (2̂4) fold difference in the amount DNA present. Moreover, these nucleic acid concentrations of the difference sample 40 types may be consistent among one another by adjusting several factors such as the amount of input lysate, bead volume, drying time and elution/dilution volumes.

The preferred device for performing the above functions of the Isolation Station 58 is a liquid handler identical in general construction to the liquid handler identified above for use as the Lysing Station 54 and the Automated Lysate Accessioning Station 56 that has been configured to automatically transfer the various reagents and other liquids as well as the magnetic particles in the manner described below. The Liquid Handler comprises a frame on which is mounted on a deck, which is surmounted by material handler, which supports and positions pipettes and is coupled to and controlled by computer, which is in turn coupled to process controller 26 to communicate information to and from LIMS 24. Liquid handler includes a syringe pump, or diluters, that are coupled to and driven by computer to dispense magnetic particles via a 16×24 array of 384 pipettes simultaneously into all 384 wells of the destination well container under the command of computer. Liquid handler may also include a second syringe pump, or diluters, that are configured to dispense a binding buffer into wells of the destination well container under computer control. The liquid handler also includes a magnet mounted on the deck, a grip or arm and a computer to move the destination well container back and forth between a first position in which the container is within the magnetic field and a second position in which the container is outside the magnetic field.

Before the functions of the Isolation Station 58 can be performed, the operator must first move the destination well container from Automated Lysate Accessioning Station 56 to deck of liquid handler and place it in a predetermined location on the deck. The destination well container from the Automated Lysate Assessing Station 56 has its barcode scanned as well as the bar-coded position on the Isolation Station 58 and recorded in the LIMS 24. Once the operator has placed the destination well container into position and scanned the barcodes, the operator starts an isolation/purification program running on computer. This program drives the operations of liquid handler causing it to dispense magnetic particles into all the wells of the destination well container containing lysed samples 40. The computer signals syringe pump to dispense the particles using pipettes into the destination well container when container is in position, away from the magnetic field created by magnet.

Once the particles have been added, computer then directs the pipettes to add a chaotropic salt, such as guadinium isothiocyanate, to each of the wells to bind the genomic nucleic acid to the magnetic particles. Once the chaotropic salt has been added, computer then mixes the contents of the wells by signaling the pipettes to alternately aspirate and redispense the material in each of the wells. This aspiration/redispensing process is preferably repeated three or four times to mix the contents in each well. In the preferred embodiment, the binding buffer is added to the lysate in the destination plate prior to the addition of the magnetically responsive beads.

Once the contents of the wells have been mixed, computer pauses for one minute to permit the particles, binding reagent, and raw biological material in the wells to incubate at room temperature. When the minute has passed, computer commands the conveyor or gripper arm to move the destination well container from one position to another position which has a magnetic field. In this position the magnet draws the magnetic particles in each of the wells downward to the bottom of the wells of the destination well container. The computer keeps the destination well container over the magnet and within the magnetic field for 2-6 minutes, or until substantially all the magnetic particles are drawn to the bottom of each well and form a small pellet.

The particles drawn to the bottom of each well have genomic nucleic acid attached to their outer surface—genomic nucleic acid that the particles hold until an elution solution is placed in each well to release the genomic nucleic acid from the particles. With the particles at the bottom of each well and the wells located within the magnetic field, the computer directs the pipettes to aspirate away the supernatant.

Once the supernatant is removed, the computer signals the conveyor or gripper arm to move the destination well container to the nonmagnetic position. The foregoing process of adding chaotropic salt, mixing the combination, pausing, drawing the magnetic particles down and aspirating the supernatant is repeated two more times.

The computer then directs the pipettes to introduce a wash solution (for example 70% ethanol to 95% ethanol) to resuspend the particles. The computer again mixes the contents of the wells by signaling the pipettes to alternately aspirate and redispense the material in each of the wells. With the wash buffer and particles thoroughly mixed, the computer again moves the destination well container back over magnet in position and draws the magnetic particles back to the bottom of the wells. This wash process is repeated to thoroughly cleanse the magnetic particles, and dilute and remove all supernatant.

Once the particles are thoroughly washed, the computer permits the magnetic particles in each well to air dry. In the preferred embodiment the operator moves the destination well container to a dryer (an ULTRAVAP dryer by Porvair Sciences, UK) having 384 tubules disposed in a 16×24 array that are configured to be simultaneously inserted into each of the wells of the destination well container and to supply warm, dry air thereto. In an alternative method, the computer causes material handler to direct compressed dry nitrogen gas into each well of the destination well container, drying the particles may or may not occur while the container is in the magnetic field. Once the particles are dried, the operator returns the destination well container to the liquid handler and directs the computer to command the pipettes to fill the wells with an elution solution and resuspend the particles. This elution solution is formulated to elute the bound genomic nucleic acid from the particles. In the preferred embodiment, the elution solution temperature is room temperature.

After resuspending the genomic nucleic acid in a solution for a predetermined period of time, the computer again moves the destination well container via conveyor or gripper arm to a position over magnet. The magnet, in turn, draws the magnetic particles down

The compounding of the primers and probes may be performed on the Sample Normalization Station 62. The stock reagents are loaded onto positions of the Sample Normalization Station. The stock reagents have their barcode numbers scanned and their position on the deck is also scanned and recorded in the LIMS 24. The computer then directs the pipettes on the Sample Normalization Station 62 to aspirate a LIMS 24 defined volume of stock reagent and dispense into a pre-capped 2-D barcoded tubes which reside in a barcoded probe box. The probe box may be vortexed to ensure proper mixing of the reagents by utilizing the T-SHAKE (TECAN, Raleigh, N.C.). The 2-D barcoded tubes are scanned with a matrix scanner and the barcode of the probe box is scanned and recorded into LIMS 24.

After the thermal cycling is completed the ABI 7900 then automatically exports the relevant files, such as the results file, the clipped file and the multicomponent file to normalization software. In the preferred embodiment the sample 40 normalization calculation is performed by a Visual Basic.net program that imports the relevant ABI files and calculates how much to dilute each individual sample 40, if any, to achieve an optimal DNA concentration.

It should be noted that the files that are imported into the normalization software also contain the data for the internal positive control (IPC). The normalization software can be configured to identify samples 40 that have IPC values that fall outside of predetermined ranges. This information is then recorded in the LIMS 24 which is configured to execute an appropriate corrective action. Alternatively, LIMS 24 can be configured to identify samples 40 that have IPC's outside of the predetermined ranges. Once the normalization software has finished calculating the appropriate dilutions for each sample 40 a dilution worklist is written such that it is importable by the liquid handler that executes the dilutions.

There are certain limitations to how much a sample 40 can be diluted because of the physical limitations available in a well plate. In such circumstances another barcoded well plate may be utilized on the deck of the Normalization Station 62. A small aliquot of the original destination plate sample 40 may be disposed into this Tertiary Destination 384 Well Container and then subsequently diluted as opposed to diluting the entire original destination plate sample 40.

The Sample Normalization Station 62 which comprises a liquid handler such as GENESIS TECAN (Raleigh Durham, N.C.) or MULTIMECK BECKMAN (Indianapolis, Ind.). It includes a frame, on which a deck is mounted to provide a horizontal working surface for original Destination 384-Well Container and/or Secondary Destination 384 Well Container and/or the Tertiary Destination 384 Well Container.

The Sample Normalization Station 62's computer is coupled to the Normalization Software such that it can import the calculated worklist. It should be noted that the Normalization Software may be also in LIMS 24. In any event the Sample Normalization Station 62's computer is also coupled to a material handler to direct the movement and operation of pipettes. Additionally, the computer is also coupled to pipettes are fluidly coupled to a syringe pump or dilutors. Once a worklist is imported the Sample Normalization Station executes the dilutions of the samples 40 via the pipettes, syringe pumps or dilutors.

The preferred device for the Amplification Plate Building Station 64 comprises a liquid handler such as Genesis TECAN (Raleigh Durham, N.C.) or Multimeck Beckman (Indianapolis, Ind.). The Amplification Plate Building Station 64 liquid handler also includes a material handler that is fixed to frame and extends upward and across the top surface of deck. A computer is coupled to material handler to direct the movement and operation of pipettes. Pipettes are fluidly coupled to a syringe pump. The appropriate destination well container is transported to the deck of the Amplification Plate Building Station 64 where its bar code is scanned. The operator places the appropriate (diluted or non-diluted) Destination 384 Well Container on a magnet, drawing all the magnetic particles to the bottom of the wells. The supernatant contains the purified genomic nucleic acid. LIMS 24 generates a worklist containing barcodes that list the STR primer components that need to be loaded onto the deck of the machine. The STR components are contained in barcoded tubes. An operator loads the barcoded tubes randomly into a primer box. The operator then scans the barcodes on the tubes using a Matrix scanner coupled to LIMS 24. Alternatively, the barcoded reagents may be loaded into a tube holder. The tube holder has bardcoded positions that are scanned and the reagent's barcodes are scanned and this correlation is recorded into LIMS 24. Further in a different embodiment the barcoded reagents may have their barcodes scanned and the regents aliquoted into a barcoded well plate, or the like. The well plate has its barcode scanned and the aliquots from the barcoded reagents are correlated to unique positions in the well plate and recorded into LIMS 24. Utilizing the aforementioned embodiments, or the like, the reagent's barcodes numbers, lot numbers and catalog numbers become part of the electronic chain-of-custody that is stored in LIMS 24.

The Amplification Plate Building Station 64 then imports the sample 40 worklist from LIMS 24. Bearing in mind that LIMS 24 is creating the sample 40 worklist based on the LIMS 24 template from the contract. Different samples 40 from different contracts may require different reagents and different reagent volumes at the Amplification Plate Building Station 64.

Once the Amplification Plate Building Station 64 imports the sample 40 worklist, and scanned the barcodes of all the reagent components, amplification plates, primary, secondary tertiary destination 384 well container(s), consumable tubes and plates and their respective barcoded positions, the automated liquid handler then executes the compounding of the STR kit constituents based on the information in the LIMS 24 worklist. Again, LIMS 24 creates the worklist which instructs the Amplification Plate Building Station 64 to compound a particular type and amount of STR reagent per kit based on the number of samples 40 in the (96 or 384) amplification plate as well as the STR reagent volume as dictated by the contract. The computer then directs the pipettes on the Amplification Plate Building Station 64 to aspirate the LIMS 24 defined volume of stock reagent or kit constituents and dispense them into 2-D barcoded pre-capped tube. The tubes are vortexed to ensure proper mixing by utilizing a T-SHAKE (TECAN, Raleigh, N.C.). The STR primer/probe combinations are contained in barcoded tubes, and in the preferred embodiment, need not be removed from the T-SHAKE. Alternatively, the STR primer/probe tubes may be created on another liquid handler such as the Probe Station. In this embodiment an operator loads the barcoded tubes into a barcoded probe box. The operator then scans the barcodes on the tubes using a Matrix scanner coupled to LIMS 24. The probe box, which is barcoded, is scanned into position on the automated Amplification Plate Building Station 64. Irrespective of the mechanism of STR primer/probe creation the important element is that the LIMS 24 and liquid handler know precisely where each STR primer/probe tube is located. In an automated fashion the liquid handler utilizes its pipettes, dilutors and/or syringe pumps to pierce the pre-slit tube cap, aspirate compounded reagent and dispense the correct number, type and volume of STR reagent into each unique utilized well of the amplification plate.

The genomic nucleic acid sample 40 from each well of the primary, secondary tertiary destination 384 well container(s) is added to a corresponding well of the amplification plate that contains the corresponding STR reagent via the automated liquid handler under the direction of the worklist vis-á-vis LIMS 24. In the preferred embodiment the plate is sealed with an automated heat sealer that consistently applies the heat sensitive seal that is amenable to piercing by liquid handlers. After the amplification plate is sealed it may or may not be vortexed to ensure maximum mixing of the nucleic acid and STR mix. If the amplification plate is vortexed it is subsequently spun a centrifuge to collect all the reaction components at the bottom of the amplification plate.

The sealed amplification plate is then placed into a thermal cycling unit, known as the Amplification Station 68. It is important to keep in mind that these standard laboratory stations are logical groupings of laboratory operations. These groupings, however, do not necessarily refer to different physical stations. Meaning, the Amplification Station 68 may physically reside on the deck of another functional Station such as the Amplification Plate Building Station 64 or maybe a standalone unit. In the preferred embodiment BIO-RAD's (HERCULES, CA) DNA Engine is utilized as the amplification station. In the most preferred embodiment a 384 thermal cycling head which accommodates a 384 well plate is used. Alternatively, a 96 thermal cycling head may be utilized. Standards thermal cycling conditions such as denaturization, cycling and hold steps are implemented.

The preferred device for performing the Capillary Electrophoresis Plate Building 73 comprises a liquid handler such as GENESIS TECAN (Raleigh Durham, N.C.) or Multimeck Beckman (Indianapolis, Ind.). It includes a frame, on which a deck is mounted to provide a horizontal working surface for first transportation apparatus and second transportation apparatus. The Capillary Electrophoresis Plate Building Station 73 liquid handler also includes a material handler that is fixed to frame and extends upward and across the top surface of deck. A computer is coupled to material handler to direct the movement and operation of pipettes. Pipettes are fluidly coupled to a syringe pump.

After thermal cycling the appropriate amplification plate is transported to the deck of the Capillary Electrophoresis Plate Building liquid station 73 handler where its bar code is scanned. In the preferred embodiment this amplification plate is placed into a transportation apparatus where its bar code is scanned as well as the barcode of the position inside the transportation apparatus and is recorded in the LIMS 24. The operator places the appropriate amplification plate, via the transportation apparatus, on the deck of the Capillary Electrophoresis Plate Building Station 73. The supernatant inside the amplification plate contains the amplified nucleic acid. LIMS 24 generates a worklist that list the Internal Lane Standards (ILS) and components that need to be loaded onto the deck of the machine. The ILS components are contained in barcoded tubes. An operator loads the barcoded tubes into it to rack. The operator then scans the barcodes on the tubes using a Matrix scanner coupled to LIMS 24. The Capillary Electrophoresis Plate Building Station 73 then imports the sample 40 worklist from LIMS 24. Bearing in mind that LIMS 24 is creating the sample 40 worklist based on the LIMS 24 template from the contract. Different samples 40 from different contracts may require different ILS reagents at the Amplification Plate Building Station 68.

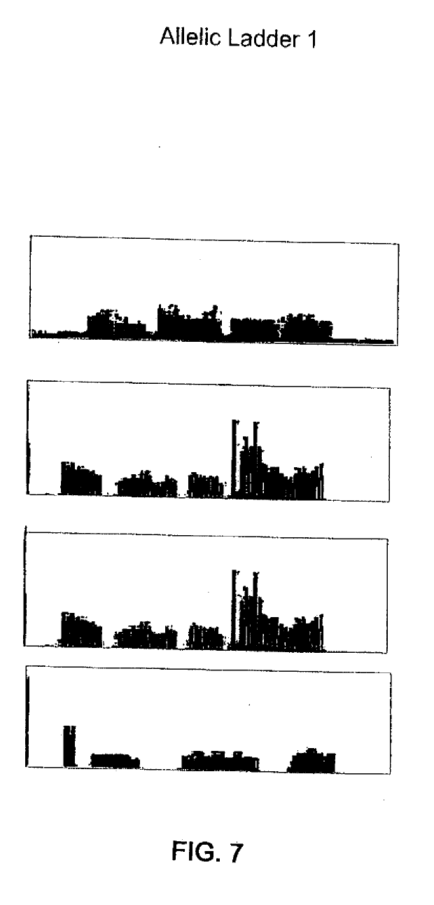

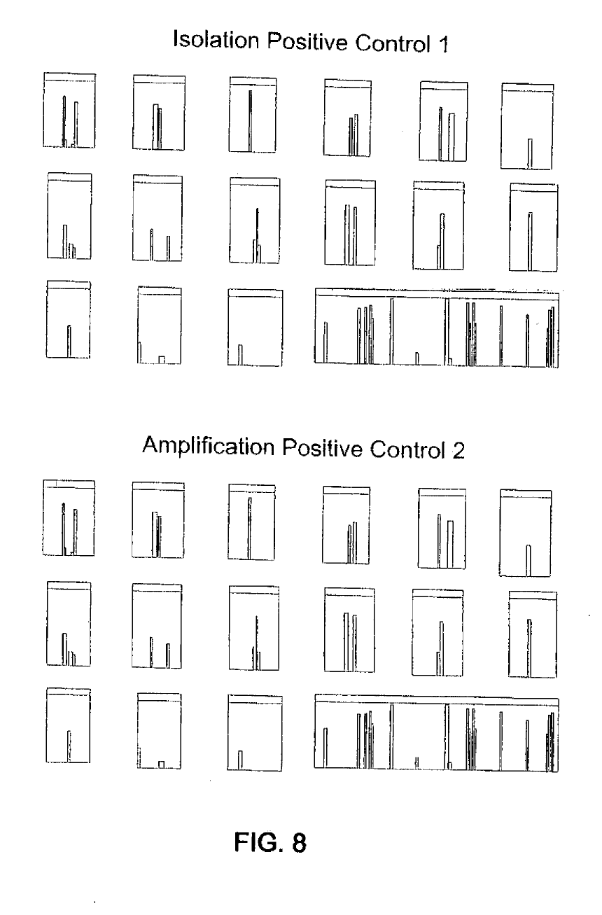

A capillary electrophoresis plate is loaded onto the deck of the Capillary Electrophoresis Plate Building liquid station 73 handler; preferably in a transportation apparatus. The capillary electrophoresis plate may be a 384 or most preferably a 96 well plate, such as those sold by APPLIED BIOSYSTEMs, (catalog numbers 4309849 and 4306737 respectively). Once the Capillary Electrophoresis Plate Building Station 73 liquid handler computer imports the sample 40 worklist, and the operator has scanned the barcodes of all the reagent components, amplification plates, capillary electrophoresis plates, consumable tubes and their respective barcoded positions, the automated liquid handler then executes the compounding of the ILS constituents based on the information in the LIMS 24 worklist. LIMS 24 creates the worklist which instructs the Capillary Electrophoresis Plate Building Station 73 to compound a particular type and amount of ILS reagent based on the number of samples 40 in the (96 or 384) amplification plate as well as the ILS reagent volume. The Capillary Electrophoresis Plate Building Station's 73 computer directs the liquid handler to utilizes its pipettes, dilutors and/or syringe pumps to aspirate and dispense the correct ILS component volumes into a pre-capped barcoded tube that resides on the Capillary Electrophoresis Plate Building Station's 733 T-SHAKE (TECAN: Raleigh, N.C.). The T-SHAKE then mixes the samples 40. The ILS combinations are contained in barcoded tubes, and in the preferred embodiment, need not be removed from the T-SHAKE. Alternatively, the ILS tubes may be created on another liquid handler such as the Probe Station. In this embodiment an operator loads the barcoded tubes into a barcoded tube rack. The operator then scans the barcodes on the tubes using a Matrix scanner coupled to LIMS 24. The tube rack, which is barcoded, is scanned into position on the automated Capillary Electrophoresis Plate Building Station 73. Irrespective of the mechanism of how the ILS was compounded the important element is that the LIMS 24 and liquid handler know precisely where each ILS tube is located. In an automated fashion the liquid handler utilizes its pipettes, dilutors and/or syringe pumps to pierce the presplit tube cap, aspirate a specified volume, and dispense the correct number, type and volume of ILS reagent into unique utilized wells of the amplification plate, including wells for isolation and amplification positive and Negative Controls and ALLELIC LADDERS as shown in FIGS. 7 and 8.

The computer then directs the pipettes on the Capillary Electrophoresis Plate Building Station 73 to pierce the heat seal of the amplification plate and aspirate the LIMS 24 defined volume of amplified DNA (typically, 1 μL to 2 μL) and dispense it into the ILS solution, which resides in the wells of the barcoded capillary electrophoresis plate. After all transfers of amplified DNA to the capillary electrophoresis plate is completed the amplification plate is re-sealed to prevent evaporation.

The barcoded capillary electrophoresis plate is then sealed as described above. The capillary electrophoresis plate is vortexed to mix the different constituents thoroughly. Subsequently, the barcoded capillary electrophoresis plate is centrifuged to pool all the reaction mixture at the bottom of each well of the plate. The amount of time and the amount of force applied to the plate need not be great in order to collect the reaction mixture at the bottom of each well. For example centrifugation for 30 seconds at 3000×g is more than enough to achieve the desired pooling. In the preferred embodiment, the capillary electrophoresis plate is then placed into a thermal cycler and denatured at 95° C. for three minutes. After the denaturization step, the plate is snap cooled on ice for three minutes. Alternatively, some laboratories may forgo the thermal denaturization step and snap cooling since formamide is a known strong denaturization agent.

The capillary electrophoresis plate is then return to the transportation apparatus, where its barcode is scanned along with the barcode of the position in the transportation apparatus, which is situated on the deck of the liquid handler. A second clean barcoded capillary electrophoresis plate has its barcode scanned along with the barcoded position in the transportation apparatus, which is also situated on the deck of the liquid handler. In the preferred embodiment, the seal that is covering the capillary electrophoresis plate containing the amplified DNA as well as the ILS, is pierced by the liquid handlers pipette tips. The liquid handler removes all the contents of each well and dispenses them into the second clean bar-coded capillary electrophoresis plate. A gray rubberized capillary electrophoresis plate mat is then applied to the capillary electrophoresis plate containing the ILS and amplified DNA.

In any event, after the gray rubberized capillary electrophoresis mat is placed on the plate, the plate is then loaded into a rigid plate base and rigid plate covering, which forms a cassette that holds the capillary electrophoresis plate during the capillary electrophoresis process. The barcode of the capillary electrophoresis plate is then scanned using a barcode scanner along with the barcoded position inside the 3130XL capillary electrophoresis machine.

LIMS 24 is configured to generate a capillary electrophoresis file which is imported into the data collection software. In the preferred embodiment, LIMS 24 creates a file that is imported from the data collection software of the 3130 XL. The worklist import contains the Container Name, the Worklist Number, the Container Type, the Application Type, the Owner, the Operator, the Application Instance, the Well, the Sample 40 Name, the Priority, the Sample 40 Type, the Analysis Method, the Panel and Bins, the Size Standard, the Results Group, and the Instrument Protocol. Once this information is imported into the data collection software the operator creates the linkage between the information and the correct plate. The operator then executes the processing of the capillary electrophoresis machine.

Upon completion of each round from the capillary electrophoresis instrument FSA or CMF files are generated for each sample 40. These FSA or CMF files are imported into one or more data 70 analysis software products (not shown). These software products include GENEMAPPER ID, GENESCAN, GENOTYPER, GENEMAPPER IDX, TRUEALLELE and FSSI³, custom designed analysis package and hybrids of each. Most of these software's work at a local computer level, however it is anticipated that these different software packages could be made available to a local area network (LAN) or a wide area network (WAN), such as the Internet. By hosting each of these products in such a format it then becomes much easier to maintain the software and make available to multiple unique users.

The vendor laboratory 20 is typically required to have two analyst independently review screening results 90 for each sample 40 to ensure accuracy. If the two vendor laboratory 20 analysts are in agreement that the sample(s) 40 are complete profiles, meaning they can be uploaded to CODIS, then the raw data 70 in the form of FSA or CMF files is transmitted to the user 1 for independent review by two additional crime laboratory analyst. If all four analysts are in agreement that the sample 40 is a complete profile it can then be uploaded to CODIS. In the event that not all of the analysts are in agreement, whether at the vendor laboratory 20 or the user 1, major and minor corrective actions will be taken to maximize the potential of producing a complete profile.

It should be noted as we described the different data reviews software options and the myriad of steps involved in processing samples 40 vis-à-vis corrective actions, data compilation and communication points between the vendor laboratory 20 and the user 1, when technically possible, these functions are automated electronic communications or templates. Specifically, files to and from instrumentation are imported and exported via a process controller 26, the instrument software, LIMS 24 or any other relational database or program. Further, in the preferred embodiment it should be noted that communication between the vendor laboratory 20 and the user 1 is done via a password-protected secure network, such as an online Order Manager 22. Alternatively, other electronic communications such as FTP sites, Internet utilities, e-mail and physical data storage devices may also be utilized.

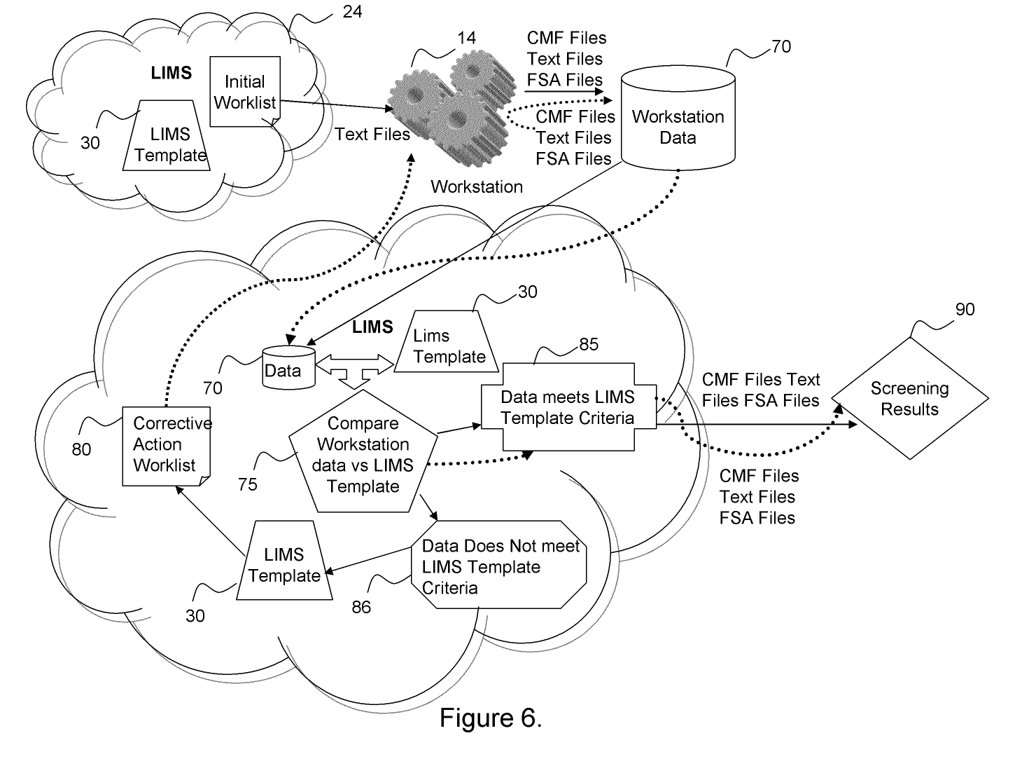

Now referring to FIG. 6, screening the samples 40 in a workstation 14 automatically directed by the template 30 while recording the sample 40 identifier at each step of the screening to generate physical data is shown. The workstation 14 data 70 is compared 75 to the template 30. If the work station data 70 from a sample 40 was found to be non-conforming 86 to the LIMS Template 30, LIMS 24 creates a corrective action work list 80 for the specific automation that is defined in the Template 30 as being appropriate for the corrective action. This corrective action work list 80 is transmitted to the workstation 14 and facilitates the rescreening of the samples 40 in a portion of the work station 14. If the data meets LIMS 24 template criteria 85 then a FSA or CMF file or text file is generated based on the physical data to provide an electronic representation of the data i.e. screening results 90. The screening results 90 can be reported to the user 1 via a web posting or as encoded in electronic media.

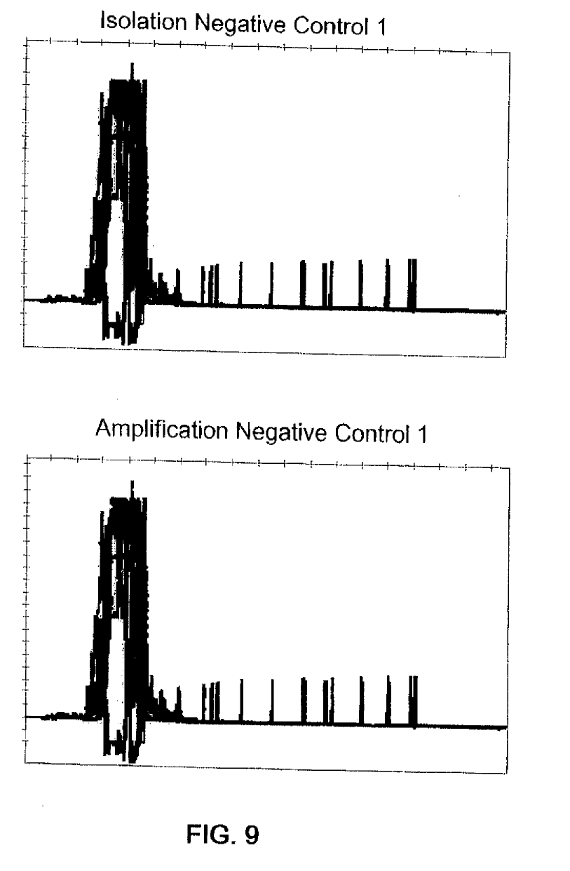

GENEMAPPER ID. The most common data analysis software currently being utilized in the user 1 is GENEMAPPER ID. The data review process begins by opening the GENEMAPPER ID software. A username and password is typically required in order to review samples 40. The operator, which in this instance is a vendor laboratory 20 analyst, will add samples 40 to the project via an import function from LIMS 24. The samples 40 are FSA or CMF files that are stored on the vendor laboratory 20's network or LIMS 24. Specifically, the imported network or LIMS 24 worklist contains the Container Name, the Worklist Number, the Container Type, the Application Type, the Owner, the Operator, the Application Instance, the Well, the Sample 40 Name, the Priority, the Sample 40 Type, the Analysis Method, the Panel and Bins, the Size Standard, the Results Group, and the Instrument Protocol. The samples 40 are imported and analyzed according to the Analysis Method that is imported into the data review software (GENEMAPPER ID) for the specific group of samples 40. Two vendor laboratory 20 analysts will begin the data review process by inspecting each peak of the ALLELIC LADDER and confirming that it matches the allele calls of the ALLELIC LADDER genotype provided by the STR kit manufacturer as shown in FIG. 7. Further, the vendor laboratory 20 analyst reviews the Isolation Positive Control and the Amplification Positive Control and confirms that the correct allele calls are present and correct as shown in FIG. 8. Moreover, the vendor laboratory 20 analyst also checks the Negative Amplification Control and Negative Isolation Control and confirms that none of these samples 40 have complete or partial profile. Moreover, the analyst will evaluate the primer peaks in the Isolation Negative Controls and Amplification Negative Controls as shown in FIG. 9. Different type of controls can be required, such as negative control, which show no peaks other than sizing ladder. The analyst must confirm that the primer peaks are present in the Negative Controls to ensure that STR reaction mix was properly added to the wells. Analysis of the unknown samples 40 then begins. The operator, which in this instance is the vendor laboratory 20 analyst, visually inspects each sample 40 looking for aberrations that will make the sample 40 ineligible for being reported to the crime laboratory 1 or CODIS.

Specifically, aberrations that commonly occur with the unknown samples 40 include no or insufficient peaks at the AMELOGEN loci, or other sex determining loci. The AMELOGEN PCR primers in the STR kit detect the X-chromosome and the Y-chromosome in humans. Clearly, no result at these loci is not possible since all humans are either genetically male or female. In such a case where no peaks are observed at the AMELOGEN loci the samples 40 must go back for a major corrective action, which entails re-isolation or re-extraction, subsequently following with re-amplification and re-injection. In the instance where the Isolation Positive Control(s) fails to produce the correct genotype the samples 40 and possibly the controls, must be re-isolated, re-extracted, and/or re-injected. When the Amplification Positive Control(s) fail to produce the correct genotype the samples 40 are typically re-amplified and/or re-injected. In instances where the Isolation Negative Control(s) yield either complete or partial profile those samples 40 will be reprocessed by re-isolating/re-extracting and/or re-injecting. Amplification Negative Controls that yield either complete or partial profiles may be rectified by re-isolating/re-extracting, re-amplifying and/or re-injecting.

Many aberrations that the analyst will identify as being not suitable for the user 1 or CODIS are not related to the Positive or Negative Controls but rather to the unknown samples 40 themselves. For instance, samples 40 that contain Off Ladder Alleles will need to have a corrective action taken such as re-amplification and/or re-injection. Some unknown samples 40 have too much DNA added to the STR reaction mix. This condition results in what is known as Pull Up. These Pull Up samples 40 are rectified by re-injecting with a lower injection time on the capillary electrophoresis instrument, re-amplifying the samples 40, or in some instances by adding less amplified PCR product in the ILS/formamide solution. Conversely, there are conditions where not enough or no DNA at all, is added to the STR reaction mix. Aberrations that occur when two little DNA is added to the STR reaction mix are known as Low Homozygotes, Low Heterozygotes, Allelic Drop Outs or No Signal. In each of these conditions the allele peaks fail to reach a predefined Relative Fluorescence Unit (RFU) threshold at, at least one or more loci. RFU is the amount of fluorescent detection an instrument can read. When these low RFU anomalies occur the samples 40 shall be reprocessed with the appropriate corrective action which may include the samples 40 being re-isolation, re-extraction, re-amplification and/or reinjection.

Some anomalies are rooted in allele frequencies. For example, microvariants are anomalies that are not necessarily incorrect genotyping but rather rarely observed alleles in the population. In such instances most crime laboratories ask/require the vendor laboratory 20 to confirm the microvariant, which may entail the sample 40 being re-isolated, re-extracted, re-amplified and/or re-injected. The specific requirement of each laboratory will be detailed in the CONTRACT which is memorialized in the LIMS 24 template for the vendor laboratory 20. In the event that the microvariant is reproduced the vendor laboratory 20 will create a microvariant table and the next time this rare allele is observed it will not need to be processed with a corrective action if it is represented on the microvariants table.

There are instances where the capillary electrophoresis instrument itself contributes artifacts that may make the data less than ideal or unsuitable. Such artifacts include Noise and Spikes. To resolve these issues usually simply re-injecting the samples 40 will suffice. In certain instances it may be beneficial to change the lot of the polymer or the electrophoresis buffer. Some anomalies such as Irregular Peak Morphology or Irregular Relative Area may be resolved by re-isolating, re-extracting, re-amplifying and/or re-injecting.

Further there are other anomalies such as Stutter may occur. Stutters is a phenomenon that occurs because the Taq polymerase slipping during amplification by one STR unit, creating a minor secondary product that is observed on the electropherogram. Stutter may be resolved by applying the corrective action of re-amplification and/or reinjection. Another category of abnormality is the Peak Height ratio (stochastic effect). The appropriate corrective action for Peak Height Ratio is re-extraction, re-isolation, re-amplification and reinjection.

The final category of abnormality that the analyst will identify is a condition known as Tri-Alleles. This is a situation where there are more than two peaks observed at a particular loci. Typically, when the analyst sees more than one peek at a locus it may strongly indicate that the sample 40 is a mixture of two or more genetic profiles. However, there are naturally occurring genetic conditions that would lead to more than one peak at a particular locus. These genetic conditions are known as trisomy's. Although relatively rare, these conditions do exist. Trisomy 21, is more commonly known as Down Syndrome. Trisomy 13 and trisomy 18 have also been observed. It should be noted that trisomy 13 and trisomy 18 are conditions that are not compatible with life. If the trisomy condition is observed/suspected the appropriate corrective action is re-isolation, re-extraction, re-amplification and/or the creation of a table.

After each of the vendor laboratory 20 analysts have manually reviewed the samples 40, looking for aforementioned anomalies, each analyst has the ability to pass or fail each individual unknown sample 40. If the vendor analyst fails the sample 40 he or she has the ability to queue that sample 40 for a major corrective action or queue the sample 40 for minor corrective action. In the preferred embodiment, this is done by having the analyst presented with a user interface that allows for the electronic identification of the sample 40 and the appropriate corrective action. In the preferred embodiment the appropriate corrective action is defined by the LIMS 24 Template 30. The information from the computer interface will be recorded and stored in the LIMS 24, written to the appropriate worklist and processed in an automated fashion on the appropriate instrumentation.

The definition of the major corrective action is that the samples 40 will be re-extracted or re-isolated. Again, this is done with the LIMS created corrective action worklist. If the samples 40 are re-isolated that means the original biological sample 40 that was received by the vendor laboratory 20, from the user 1, will be re-sample 40 and reprocessed from the beginning with the automation. If the sample 40 is re-extracted that means the lysate created from the original sample 40 will be re-process with the automation. Depending on whether the sample 40 is re-extracted or re-isolated most, if not all, of the instrumentation will be utilized with this type of corrective action.

A minor corrective action is defined as simply re-injecting the samples 40 with the automated capillary electrophoresis instrument or Enriching/Spiking the ILS/formamide solution with more amplified product from the corresponding sample 40 from the amplification plate. Minor corrective actions typically involve liquid handlers such as the Amplification Plate Building Station 64/the Capillary Electrophoresis Plate Building Station 73 or the capillary electrophoresis instrumentation (i.e. 3130XL).

When the samples 40 are reprocessed with a minor corrective action LIMS 24 applies the considerations (LIMS 24 Template 30) for the reprocessing of the samples 40. Additionally, LIMS 24 creates a linkage between the data of the initial sample 40 processing as well as the data generated from the application of the minor corrective action. The sets of data are linked together for traceability and review. At the completion of the minor corrective action the automated capillary electrophoresis instrumentation creates the FSA or CMF files. The FSA or CMF files and associated files are imported into the GENEMAPPER ID software as described above.

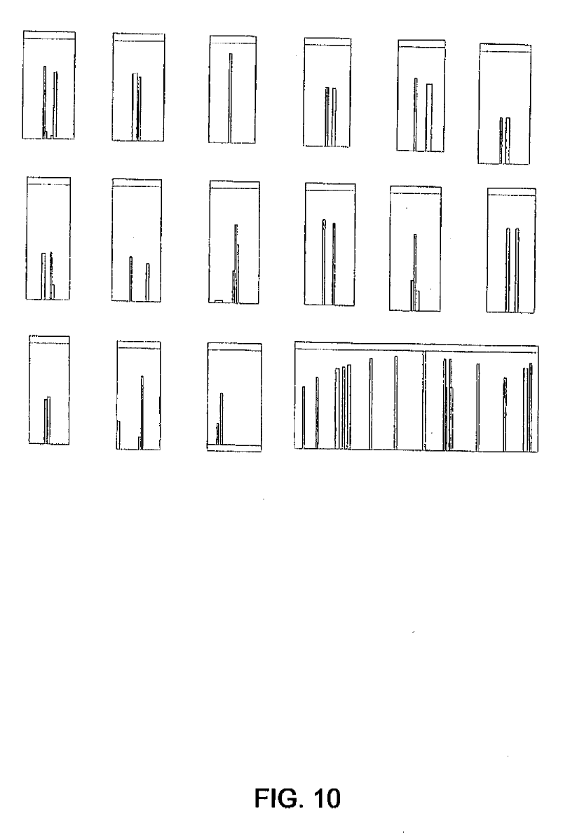

The samples 40 are then reviewed by two vendor analyst by using the GENEMAPPER ID software as shown in FIG. 10. These may be the same two vendor analyst that reviewed the original data or alternatively it could be different analyst altogether. The vendor analysts will then review the aforementioned anomalies that occur with the electropherogram. These anomalies include evaluation of the AMELOGEN, the Isolation Positive and Negative Controls, the Amplification Positive and Negative Controls, the Negative Controls Primer Peaks, Off Ladder Alleles, Pull Up, low overall Signal Height, (i.e. Low Homozygous Contracts, Low Heterozygotes Contracts and no signal) Microvariants, Noise, Peak Morphology, Relative Area, the Peak Height Ratio, Stutter, and Tri-alleles. The remedies for each of these anomalies are described above. Different type of controls are required, such as a negative control, which should sow no peaks other that the sizing ladder. If a peak is seen the contamination is present somewhere in the workstation 14. The Amplification positive control DNA of know genotype that must produce the correct genotyping result or there is an issue in the workstation 14

Upon review of each of the samples 40 that have had a minor corrective action applied the analyst will either pass or fail for a second time the samples 40. If the minor corrective action resolves the anomaly each analyst will then pass that particular sample 40 or group of samples 40. Upon completion of the sample 40 or a subset of samples 40, the FSA or CMF files, the Analysis Method, the Panels and Bins, will be exported by the capillary electrophoresis instrument and compiled with the original FSA or CMF files, the original Analysis Method, and the original Panels and Bins in LIMS 24. LIMS 24 will compile the original sample 40 data as well as the rerun and corrected sample(s) 40 data and pertinent data such as all the ALLELIC LADDERS and Positive and Negative Controls associated with both of those sample 40 runs for the crime laboratories inspection and review.

The vendor laboratory 20 analyst will then run a program that checks and compares the vendor laboratory 20 staff's profiles against the profiles (FSA or CMF file) of that particular project or run. This prevents any inadvertent laboratory cross-contamination from reaching the user 1 and potentially being loaded into CODIS.

The vendor laboratory 20 will transmit the files in electronic format to the user 1. This electronic communication includes e-mail, FTP sites, local area networks (LAN) and wide area networks (WAN), the Internet, as well as any other physical data media such as a CD, jump drives, or any other disk. In many cases the user 1 requests/requires the information to be written onto a compact disk (CD). The CD contains the FSA or CMF files as well as all other associated FSA or CMF file, Analysis Method, Panels and Bins, etc. of the original and reworked samples 40. The data is then received by the user 1 in a format that will be reviewed by two crime laboratory analysts.

In the event that a sample 40 or a group of samples 40 fails the minor corrective action the analyst will electronically document the samples 40 at a user interface, which will be recorded in the LIMS 24. In the event that the minor corrective action does not rectify the samples 40 the operator, who is the vendor laboratory 20 analyst in this case, will then electronically queue these samples 40 for the appropriate major corrective action.

Samples 40 that fail either the laboratory analyst's initial review or if a minor corrective action fails to correct the samples 40 or group of samples 40 then a major corrective action will be implemented to maximize the potential for developing a complete profile. The samples 40 are electronically queued for a major corrective action will still be evaluated with the LIMS Template 30 as was the initial evaluation. Meaning, that the samples 40 will be reevaluated with the same Analysis Method that the original samples 40 were evaluated. Upon completion of the major corrective action, which is usually re-extraction and/or re-isolation, re-amplification, and reinjection, the samples 40 FSA or CMF and associated files will then be imported into the GENEMAPPER ID software program as described above. The vendor laboratory 20 analyst will then review the samples 40 to identify anomalies that will not be permitted by the user 1 or up loadable to CODIS. These anomalies, again, include evaluation of the AMELOGEN, the Isolation Positive and Negative Controls the Amplification Positive and Negative Controls, Negative Control Primer Peaks, Off Ladder Alleles, Pull Up, low overall Signal Height, (i.e. Low Homozygous Contracts, Low Heterozygotes Contracts and no signal) Microvariants, Noise, Peak Morphology, Relative Area, the Peak Height Ratio, Stutter, and Tri-alleles. If either vendor laboratory 20 analyst finds anomalies and fails the sample 40 for a second time (initial processing and with a major corrective action) that sample 40 will be noted in LIMS 24. Since the sample 40 has failed twice, typically the vendor laboratory 20 can invoice the user 1 for the sample 40 even though no acceptable profile was generated by the vendor laboratory 20. By utilizing the data management features of the LIMS 24, the data from the initial run and the rerun(s) is compiled into a technical file and sent to the user 1 as a unique technical file. Contained in this technical file are the FSA or CMF files (the Samples 40 and Positive and Negative Controls) the Panels, the Bins, the ALLELIC LADDERS and the Analysis Method. LIMS 24 also generates a non-technical report for the user 1 showing which samples 40, although being run twice, failed for accounting purposes.

In the event that the vendor analyst does not identify any anomalies after a major corrective action, (either directly from the original analysis to a major corrective action or a major corrective action administered after the failure of a minor corrective action resolving the anomaly) then the acceptable data will be managed by LIMS 24. Again by utilizing the LIMS 24 data management functionality the original and rerun FSA or CMF files (the Samples 40 and Positive and Negative Controls), the Panels, the Bins, the ALLELIC LADDERS, Instrument Protocol and the Analysis Method are compiled. The capillary electrophoresis instrument exports the appropriate files for the compilation. LIMS 24 may then in turn export or write the aforementioned files to another location such as a database, a standalone program (i.e. that compares FSA or CMF files) or a network. The completed unknown genetic profiles, formatted as FSA or CMF files, that are being prepared to be sent to the user 1 are then compared against the known genetic profiles of the vendor laboratory 20 staff. This ensures that no inadvertent cross-contamination occurred leading to false data being sent to the user 1 and potentially uploaded to the CODIS database. The completed files are then transmitted to the user 1 via any electronic communication. This electronic communication includes e-mail, FTP sites, local area networks (LAN) and wide area networks (WAN), the Internet, as well as any other physical data media such as a CD, some drive, or any other disk.

Sample 40 data may be received by the user 1 after it was determined that complete profiles were ascertained via review by two vendor laboratory 20 analyst from either the original sample 40 processing, the original sample 40 run plus a minor corrective action or the original sample 40 run plus a major corrective action with or without a preceding minor corrective action. If the sample 40 passed the vendor laboratory 20 analyst at any time then the sample FSA or CMF files (including Positive and Negative Controls and in addition to ALLELIC LADDERS), the Analysis Method (LIMS Template 30), the Panels and the Bins will be exported from the capillary electrophoresis instrument to the LIMS 24 database. The vendor laboratory 20 analyst will then run a program that checks and compares the vendor laboratory 20 staff's profiles against the profiles (FSA or CMF file) of that particular project or run. This prevents any inadvertent laboratory cross-contamination from reaching the user 1 and potentially being loaded into CODIS. The vendor laboratory 20 will transmit the files in electronic format to the user 1. This electronic communication includes e-mail, FTP sites, local area networks (LAN) and wide area networks (WAN), the Internet, as well as any other physical data media such as a CD, some drive, or any other disk.

The data is then received by the user 1 in a format that will be reviewed by two crime laboratory 1 analysts. In this example a crime lab is also utilizing GENEMAPPER ID as the data review software. The crime laboratory analyst imports the files received from the vendor laboratory 20 into the GENEMAPPER ID software. This imported information includes the FSA or CMF files, the Analysis Method, the Panels in the Bins. After each of the analysts have manually reviewed the samples 40, looking for aforementioned anomalies, each analyst has the ability to pass or fail each individual unknown sample 40, Positive Controls, Negative Controls and ALLELIC LADDERS.

The vast majority of samples 40 that are received from the vendor laboratory 20 should pass the analysts technical review with little or no rejections. If the samples 40 are found to be compliant with the contract requirements then the crime laboratory analyst will accept the samples 40. After accepting the samples 40 as being compliant with the contract the user 1 may randomly check the FSA or CMF file that will ultimately be uploaded to CODIS. The screening results 90 are now ready to be uploaded into the CODIS database. The uploading of profiles into the CODIS database can only be done by the CODIS administrator at the user 1. This database is hosted and administered by the Federal Bureau of Investigation (FBI). After it is determined that the samples 40 are acceptable for CODIS upload, the user 1 communicates with the vendor laboratory 20 that the samples 40 were acceptable. Preferably this communication is done in electronic form. The vendor laboratory 20 upon receiving confirmation that the samples 40 were acceptable by the user 1 the vendor laboratory 20 will then generate an invoice that is presented to the user 1.

If the analyst fails the sample 40 the analyst has the ability to electronically communicate to the vendor laboratory 20 samples 40 identification failed and the reason for the failure, i.e. “sample failure identification.” The vendor laboratory 20 will then rescreen that specific sample(s) 40 with the appropriate major corrective action in the workstation 14. Upon completion of the major corrective action the sample(s) 40 will then be reviewed manually by two analysts. The samples 40 that were reprocessed with a major corrective action will still be evaluated with the LIMS 24 template (contract considerations) as was the initial evaluation. Meaning, that the samples 40 will be reevaluated with the same Analysis Method as the original Analysis Method. Upon completion of the major corrective action, which is usually re-extraction and/or re-isolation, re-amplification, and reinjection, the samples 40 FSA or CMF and associated files will then be imported into the GENEMAPPER ID software program, as described above, at the vendor laboratory 20 analyst computer. The vendor laboratory 20 analyst will then review the samples 40 to identify anomalies that will not be permitted by the user 1 or up-loadable to CODIS. These anomalies, again, include evaluation of the AMELOGEN, the Isolation Positive and Negative Controls the Amplification Positive and Negative Controls, Negative Control Primer Peaks, Off Ladder Alleles, Pull Up, low overall Signal Height, (i.e. Low Homozygous Contracts, Low Heterozygotes Contracts and no signal) Microvariants, Noise, Peak Morphology, Relative Area, the Peak Height Ratio, Stutter, and Tri-alleles.

If either vendor laboratory 20 analyst finds anomalies and fails the sample 40 for a second time (initial processing and a major corrective action) that sample 40 will be noted in LIMS 24. Since the sample 40 has failed twice, typically the vendor laboratory 20 can invoice the user 1 for the sample 40 even though no acceptable profile was generated by the vendor laboratory 20. By utilizing the data management features of the LIMS 24, the data from the initial run and the rerun is compiled into a technical file and sent to the user 1 as a unique technical file. Contained in this technical file are the FSA or CMF files (the Samples 40 and Positive and Negative Controls) the Panels, the Bins, the ALLELIC LADDERS and the Analysis Method. LIMS 24 also generates a non-technical report for the user 1 showing which samples 40, although being run twice, failed for accounting purposes.

Alternatively, if the sample 40 initially passed the two vendor laboratory 20 analysts, was rejected by one of the analyst and then subsequently failed upon the vendor laboratory 20 analyst review after the major corrective action; the sample 40 may then be retested with an additional major corrective action. Upon completion of the second major corrective action the sample(s) 40 will then be imported and reviewed manually by two vendor laboratory 20 analysts. The samples 40 that were reprocessed with the second major corrective action will still be analyzed with the LIMS 24 template as was the initial evaluation. Upon completion of the second major corrective action, which is usually re-extraction and/or re-isolation, re-amplification, and reinjection, the samples 40 FSA or CMF and associated files will then be imported into the GENEMAPPER ID software program, as described above, at the vendor laboratory 20 computer. The vendor laboratory 20 analyst will then review the samples 40 to identify anomalies that will not be permitted by the user 1 or uploadable to CODIS. These anomalies, again, include evaluation of the AMELOGEN, the Isolation Positive and Negative Controls, the Amplification Positive and Negative Controls, Negative Control Primer Peaks, Off Ladder Alleles, Pull Up, low overall Signal Height, (i.e. Low Homozygous Contracts, Low Heterozygotes Contracts and no signal) Microvariants, Noise, Peak Morphology, Relative Area, the Peak Height Ratio, Stutter, and Tri-alleles. If either vendor laboratory 20 analyst finds anomalies and fails the sample 40 for a third time (initial processing and with a two major corrective actions) that sample 40 will be noted in LIMS 24. Since the sample 40 has failed thrice, typically the vendor laboratory 20 can invoice the user 1 for the sample(s) 40 even though no acceptable profile was generated by the vendor laboratory 20. By utilizing the data management features of the LIMS 24, the data from the initial run and the reruns is compiled into a technical file and sent to user 1 as a unique technical file. Contained in this technical file are the FSA or CMF files (the Unknown Samples 40 and Positive and Negative Controls) the Panels, the Bins, the ALLELIC LADDERS and the Analysis Method. LIMS 24 also generates a nontechnical report for the user 1 showing which samples 40, although being run twice, failed for accounting purposes.

If the second major corrective action resolves the anomaly each vendor laboratory 20 analyst will then pass that particular sample 40 or group of samples 40. Upon completion of the sample 40 or a subset of samples 40, the FSA or CMF files (Samples 40, Positive and Negative Controls, ALLELIC LADDERS), the Analysis Method, the Panels and Bins, the re-runs of the FSA or CMF files, the re-runs of the Analysis Method, and the re-runs of the Panels and Bins will be/or have been exported by the capillary electrophoresis instrument and compiled in LIMS 24. LIMS 24 will compile the original sample 40 data as well as the reruns (corrected sample(s) 40 data) and all pertinent data files associated with those sample 40 runs for the crime laboratories inspection and review. The vendor laboratory 20 analyst will then run a program that checks and compares the vendor laboratory 20 staff's profiles against the profiles (FSA or CMF file) of that particular project or run. This prevents any inadvertent laboratory cross-contamination from reaching the user 1 and potentially being loaded into CODIS. The vendor laboratory 20 will transmit the files in electronic format to the user 1. This electronic communication includes e-mail, FTP sites, local area networks (LAN) and wide area networks (WAN), the Internet, as well as any other physical data media such as a CD, some drive, or any other disk.

The data is then received by the user 1 in a format that will be reviewed by two analysts. In many cases the crime laboratories request/require the information is written onto a CD. The CD contains the FSA or CMF files as well as all the other associated FSA or CMF, Analysis Method, Panels and Bins of the original and reworked samples 40. The samples 40 on the CD are now ready to be reviewed by the two crime laboratory analysts.

Some samples 40 may be found to be compliant with the contract requirements after initially passing the vendor laboratory 20 analyst review process, failing the crime laboratory 1 analyst data review and then subsequently passing the vendor laboratory 20 analyst data review after a major corrective action. If the post crime laboratory analyst rejection major corrective action resolves the anomaly each vendor laboratory 20 analyst will then pass that particular sample 40 or group of samples 40. Upon completion of the sample 40 or a subset of samples 40, the FSA or CMF files, the Analysis Method, the Panels and Bins, the re-runs of the FSA or CMF files, the re-runs of the Analysis Method, and the re-runs of the Panels and Bins will be or have been previously exported by the capillary electrophoresis instrument and compiled in LIMS 24. LIMS 24 will compile the original sample 40 data as well as the reruns (corrected sample(s) 40 data) and all pertinent data files associated with those sample 40 runs for the crime laboratories inspection and review. The vendor laboratory 20 analyst will then run a program that checks and compares the vendor laboratory 20 staff's profiles against the profiles (FSA or CMF file) of that particular project or run. This prevents any inadvertent laboratory cross-contamination from reaching the user 1 and potentially being loaded into CODIS. The vendor laboratory 20 will transmit the files in electronic format to the user 1. This electronic communication includes e-mail, FTP sites, local area networks (LAN) and wide area networks (WAN), the Internet, as well as any other physical data media such as a CD, jump drives, or any other disk. The data is then received by the user 1 in a format that will be reviewed by two crime laboratory analysts. In many cases the user 1 may request/require the information to be written onto a CD. The data is then received by the user 1 in a format that will be reviewed by two crime laboratory analyst.

In this example a user 1 is using GENEMAPPER ID as the data review software. The crime laboratory analyst imports the results 90 files received from the vendor laboratory 20 into the GENEMAPPER ID software. This imported information includes the FSA or CMF files, the Analysis Method, the Panels and Bins, Protocol etc. After each of the user 1 analysts have manually reviewed the samples 40, looking for aforementioned anomalies, each analyst has the ability to pass or fail each individual unknown sample 40.

The vast majority of samples 40 that are received from the vendor laboratory 20 should pass the crime laboratory analyst technical review with little or no rejections. If the samples 40 are found to be compliant with the contract requirements then the analyst will accept the samples 40. After accepting the samples 40 as being compliant with the contract the user 1 may randomly check the FSA or CMF file that will ultimately be uploaded to CODIS. The user 1 may employ manual techniques or software techniques to randomly check the FSA or CMF file. The samples 40 are now ready to be uploaded into the CODIS database. The uploading of profiles into the CODIS database can only be done by the CODIS administrator at the user 1. This database is hosted and administered by the Federal Bureau of Investigation (FBI). After it is determined that the samples 40 are acceptable for CODIS upload, the user 1 communicates with the vendor laboratory 20 that the samples 40 were acceptable. Preferably this communication is done in electronic form.

If the crime laboratory analyst fails the sample 40 the user 1 communicates to the vendor laboratory 20 specifically what samples 40 failed and the reason why each of the samples 40 failed. By using the data management features of the LIMS 24, the data 70 from the initial run and the reruns are compiled into a technical file and sent to the user 1 as a unique technical file. Contained in this technical file are the FSA or CMF files (the Samples and Positive, Negative Controls, ALLELIC LADDERS etc.) the Panels, the Bins, the ALLELIC LADDERS and the Analysis Method.

Vendor Web Enabled Review. In one embodiment, the DNA sample review process may occur in a secure Internet enabled environment. Meaning that the vendor laboratory 20 as well as a user 1 would log into a secure site which would enable both parties to seamlessly review, except, reject, or retest samples 40 from a single interface.

The process begins with the physical processing of the samples 40 at the vendor laboratory 20. The samples 40 are added to the data collection template for processing by the capillary electrophoresis instrument. The sample 40 review will be done manually by two vendor laboratory 20 analyst. The information that is produced by the Capillary Electrophoresis instrument, such as the FSA or CMF files, the analysis method, the panels, are reviewed in a software environment that is hosted on servers that are accessible from the World Wide Web or any other electronic communication. The vendor laboratory 20 analyst reviews each sample 40 utilizing the review software looking for the anomalies including the evaluation of the AMELOGEN, the Isolation Positive and Negative Controls the Amplification Positive and Negative Controls, Negative Control Primer Peaks, Off Ladder Alleles, Pull Up, low overall Signal Height, (i.e. Low Homozygous Contracts, Low Heterozygotes Contracts and no signal) Microvariants, Noise, Peak Morphology, Relative Area, the Peak Height Ratio, Stutter, and Tri-alleles. The vendor analysts each independently review the samples 40 on the vendor servers. At this web interface the vendor analyst are able to choose the ALLELIC LADDERS from a drop-down feature. This drop-down feature has a list of predefined ALLELIC LADDERS. By choosing the ALLELIC LADDER at the web interface it enables the software to compare against the ALLELIC LADDER from the project. This confirmation will either conform to the chosen ALLELIC LADDERS or it will indicate that there is an issue with the particular ALLELIC LADDERS from the specific project. Additionally, at the web interface the analyst is able to choose and confirm the Isolation Positive Controls, the Amplification Positive Control, the Isolation Negative Control, and the Amplification Negative Control. Each of these Controls and Ladders may have a number of possibilities depending on the type of STR kit used and the type of controls used. For instance many laboratories will use samples 40 collected from individuals from the laboratory staff to serve as an Isolation Positive Controls, so there may be multiple Positive Controls that can be selected for a particular project.

After the analyst selects the appropriate Ladders and Controls the software performs a Ladder control check that compares and confirms the analyst chosen Ladders and Controls against the allele calls of the projects Ladders and Controls. If the allele calls match the particular Controls and/or ALLELIC LADDER the software then visually represents to the analyst that there was an exact match. This can be done with the indicator flags by simply using color, fonts and pop-ups to identify the match. In the same way if there is a mismatch between the analysts selected ALLELIC LADDERS and Controls and the project's ALLELIC LADDERS and controls then the software visually represents these issues in such a way that it notifies the analyst and requires attention. At the end of the ALLELIC LADDER and Control check the vendor laboratory 20 analyst has the ability to accept, reject or edit and comment the ALLELIC LADDERS, the Negative Controls, and the Positive Controls.

After the vendor analyst confirms that the ALLELIC LADDERS, the isolation Negative Controls, the amplification Negative Controls, the isolation Positive Controls, and the amplification positive control were acceptable in the software performs the allele calls on the unknown samples 40. At this point the analyst has the ability to make notes that will be captured and displayed to the other vendor analyst as well as a crime laboratory analyst. The two vendor laboratory 20 analyst then independently review each individual samples 40 according to the DNA database and guidelines and are parsed into accepted and rejected categories.

At the vendor laboratory 20 analyst agree upon the allele calls after independent sample 40 review. The allele calls are then compared against the vendor laboratory 20 staffs allele calls, also known as the staff's genetic profiles. At this point the unknown samples 40 are added to the user 1's account, which is hosted and accessible remotely through a method such as the World Wide Web. Information that is added to the user l's account is the raw information that is needed to perform review by the user 1. This information includes the projects FSA or CMF files, Analysis Method, the Panels & Bins, etc. At this point an e-mail notification is sent to the user 1 or in one embodiment directly to the crime laboratory analyst. This electronic e-mail notification indicates that the samples 40 (project) are ready for the user 1 review.

The crime laboratory analyst would then log into their account via secure connection. Each analyst has a unique identification and password that is recorded for the electronic Chain-of-Custody. Each crime laboratory analyst independently opens the project which contains the unknown samples 40 in the Web enabled environment. The analyst independently select the ALLELIC LADDERS, the isolation negative Control, the Isolation Positive Control, the Amplification Negative Control and the Amplification Positive Control. In the same manner as described above the software will indicate, using indicator flags, to the crime laboratory analyst if there was a match or mismatch between the ALLELIC LADDERS and Controls of the user settings and the ALLELIC LADDERS, Controls and allele calls from the project itself.

After the crime laboratory analyst confirms that the allele calls and Positive/Negative Controls were acceptable the analyst is presented with the allele calls of the unknown samples 40. At this point the crime laboratory analyst has the ability to create notes or review notes from the vendor analyst. The loci and peaks are laid out in such a way that the user can visually see all the loci and indicator flags in one screen view. The indicator flags are the visual elements that identify the loci that met the analysis method parameters. The loci in this view can be double clicked to zoom in for more detailed inspection of the electropherogram for that particular loci. Moreover, if two STR kits are utilized for a single sample 40, such as in the case of Profiler Plus and Cofiler both kits internal lane standards (ILS) and duplicated loci are presented on one screen with special indication to the loci overlap.

The two crime laboratory analysts have the ability to independently accept, hold or retest each individual sample 40 based on the DNA database guidelines. It should be noted that if one of the loci has a particular issue from the DNA template for Analysis Method, that particular loci will be indicated in such a way to draw the analyst attention. This can be done in a number of ways utilizing indicator flags, such as highlighting the loci or coloring in such a way to draw the user's attention. The analyst has the ability to agree with the two vendor analyst, hold those particular samples 40 for a later decision or queue samples 40 for a retest.

The crime laboratory analyst has the ability to accept the sample 40 or retest the sample 40. This process is done utilizing a confirmation page. This confirmation page shows the samples 40 that were held previously and gives the option to review those samples 40 and accept the samples 40. Additionally on this page is a list of samples 40 that are to be retested. There is the option to cancel the retest and accept a profile. Once the crime laboratory analyst configures the held samples 40 and retested samples 40 the project is either confirmed, which will execute the retesting, or the entire project is rejected, which re-queues it for technical review by the crime laboratory analyst.

The samples 40 that are approved by the crime laboratory analyst agree with the vendor laboratory 20 analyst. The user 1 may choose to randomly check the FSA or CMF file generated by the software. At this point the data including the CMF files, the FSA, the Analysis Method, the Panels & Bins, etc. are transmitted to be user 1 via any electronic format. Many crime laboratories require a physical CD of the electronic data. The FSA or CMF files are then ready to be uploaded to the CODIS. By the user 1 accepting the samples 40 and/or transmitting the data from the excepted samples 40 the vendor laboratory 20 LIMS 24 creates a report which includes an invoice that is presented to the user 1 detailing the acceptable samples 40.

If the crime laboratory analysts determine that the samples 40 are to be retested then the crime laboratory analyst will identify the reason that a specific sample 40 is to be retested. The sample 40 is retested by the vendor laboratory 20 according to the LlMS Templates Corrective Action 75. By the crime laboratory analyst confirming the retest of a particular sample 40 on the Web enabled interface, it prompts the LIMS 24 to create a worklist that is received by the appropriate instrument at the vendor laboratory 20 at the proper place in the workflow. The original samples FSA or CMF files, Controls, ALLELIC LADDERS, Analysis Method, Panels & Bins, etc. are linked to the retested samples controls, ALLELIC LADDERS, Analysis Method, Panels & Bins, etc. in the LIMS 24.

At this point the vendor laboratory 20 analysts independently review the retested samples 40 on the vendor enabled web accessible server. The vendor analyst independently selects the ALLELIC LADDERS, the Isolation Negative Control, the Isolation Positive Control the Amplification Negative Control and the Amplification Positive Control. In the same manner as described above the software will indicate, using indicator flags, to the vendor laboratory 20 analysts if there was a match or mismatch between the ALLELIC LADDERS and Controls of the user settings and the ALLELIC LADDERS, Controls and allele calls from the project itself.

After the vendor analyst confirms that the ALLELIC LADDERS, the Isolation Negative Controls, the Amplification Negative Controls, the Isolation Positive Controls, and the Amplification Positive Control were acceptable in the software performs the allele calls on the unknown samples 40. At this point the analyst has the ability to make notes that will be captured and displayed to the other vendor analyst as well as a crime laboratory analyst. The two vendor laboratory 20 analyst then independently review each individual samples 40 according to the DNA database guidelines and are parsed into accepted and rejected categories.

If the vendor laboratory 20 analysts agree upon the allele calls after independent sample 40 review then the allele calls are compared against the vendor laboratory 20 staffs allele calls, also known as the staff's genetic profiles. At this point the unknown samples 40 are added to the user 1's account which is hosted and accessible remotely through a secure method such as the World Wide Web. Information that is added to the user 1's account is the raw information that is needed to perform review by the user 1. This information includes the projects FSA or CMF files, Analysis Method, the Panels & Bins, etc. At this point an e-mail notification is sent to the user 1 or in one embodiment directly to the crime laboratory analyst. This electronic e-mail notification indicates that the samples 40 (project) are ready for the user 1 review.

The crime laboratory 1 analyst would then log into their account via secure connection. Each analyst has a unique identification and password that is recorded for the electronic Chain-of-Custody. Each crime laboratory analyst independently opens the project which contains the unknown samples 40 in the Web enabled environment. The crime laboratory analyst independently select the ALLELIC LADDERS, the Isolation Negative Control the Isolation Positive Control the Amplification Negative Control and the Amplification Positive Control. In the same manner as described above the software will indicate, using indicator flags, to the crime laboratory analyst if there was a match or mismatch between the ALLELIC LADDERS and Controls of the user settings and the ALLELIC LADDERS and Control allele calls from the project itself.

After the crime laboratory analyst confirms that the allele calls of the Positive and Negative Controls were acceptable the crime laboratory analyst is presented with the allele calls of the unknown samples 40. At this point the crime laboratory analyst has the ability to create notes or review notes from the vendor analyst. The loci and peaks are laid out in such a way that the user can visually see all the loci and indicator flags on one screen view. The indicator flags are the visual elements that identify if the loci met or did not meet the Analysis Method parameters. The loci in this view can be double clicked to zoom in for more detailed inspection of the electropherogram for a particular locus. Moreover, if two STR kits are utilized for a single sample 40, such as in the case of PROFILER PLUS and COFILER both kits Internal Lane Standards (ILS) are presented on one screen with special indication to the loci overlap.

The two crime laboratory analysts have the ability to independently accept or reject each individual sample 40 based on the DNA database guidelines. It should be noted that if one of the loci has a particular issue from the LIMS 24 template or Analysis Method, that particular loci will be indicated in such a way to draw the analyst attention. This can be done in a number ways utilizing indicator flags such as highlighting the loci or coloring in such a way to draw the user's attention.

The samples 40 that are approved by the crime laboratory analyst agree with the vendor laboratory 20 analyst. The user 1 may choose to randomly check the FSA or CMF file generated by the software. At this point the data including the CMF files, the FSA, the Analysis Method, the Panels and Bins, etc. are transmitted to be user 1 via any electronic format. Many crime laboratories require a physical CD of the electronic data. The FSA or CMF files are then ready to be uploaded CODIS.

Alternatively, the crime laboratory analyst may reject the samples 40 even after the retest. The crime laboratory analyst identifies specifically which samples 40 are to be rejected and the reason each sample 40 was rejected. The original sample 40 data including the FSA or CMF files, the Controls, the ALLELIC LADDERS, the Analysis Method, and the Panels and Bins, etc. are linked and compiled in the vendor's LIMS 24 with the retested sample 40 information including the retested FSA or CMF files, Controls, ALLELIC LADDERS, the Analysis Method, Panels and Bins, etc. This information is transmitted to the user 1 via electronic communication. In many instances this information is reduced to CD and sent to the user 1.

Depending on the reason the vendor laboratory 20 analyst failed the sample 40 will determine which type of corrective action should be implemented. Minor corrective actions are defined as samples 40 that are reprocessed with a spiking/enriching of the amplified product and/or a reinjection of the sample 40 on the capillary electrophoresis instrument. Once it is determined that a sample 40 is to be retest with a minor corrective action the LIMS 24 writes a worklist for that specific samples 40 and that specific sample 40 is retested by the vendor laboratory 20 in the appropriate place in the production line. It should be noted that the vendor laboratory 20 LIMS 24 which links compiles and contains the FSA or CMF files, the controls, the ALLELIC LADDERS, Analysis Method, Panels and Bins, etc. of the original run is also recording the same information for the retested sample 40.

At this point the vendor laboratory 20 analyst, which may be the same analyst or different, will then review the samples 40 on the secure Web-enabled software. The vendor laboratory 20 analyst independently selects the ALLELIC LADDERS, the Isolation Negative Control, the Isolation Positive Control, the Amplification Negative Control and the Amplification Positive Control. In the same manner as described above the software will indicate, using indicator flags, to the crime laboratory analyst if there was a match or mismatch between the ALLELIC LADDERS and Controls of the user settings and the ALLELIC LADDERS and controls allele calls from the project itself.

After the vendor analyst confirms that the ALLELIC LADDERS, the Isolation Negative Controls, the Amplification Negative Controls, the Isolation Positive Controls, and the Amplification Positive Control were acceptable the software performs the allele calls on the unknown samples 40. At this point the analyst has the ability to make notes that will be captured and displayed to the other vendor analyst as well as a crime laboratory analyst. The two vendor laboratory 20 analyst then independently review each individual samples 40 according to the DNA database guidelines and are parsed into accepted and rejected categories.

In the event that the vendor analysts pass the samples 40 then all of the original information and all the retested information is compiled. Meaning, that the original samples 40 FSA or CMF files, Controls, ALLELIC LADDERS, Analysis Method, Panels and Bins, etc. as well as the retest FSA or CMF Files, Controls, ALLELIC LADDERS, Analysis Method, Panels and Bins, etc. will be associated with that particular sample 40 for other vendor and crime laboratory analyst review. Once the vendor laboratory 20 analyst agree upon the allele calls after independent sample 40 review then the allele calls are compared against the vendor laboratory 20 staffs allele calls, also known as the staff's genetic profiles. At this point the unknown samples 40 are added to the user 1's account which is hosted and accessible remotely through a secured method such as the World Wide Web. The information that is added to the crime laboratory 1's account is the raw information that is needed to perform review by the user 1. This information includes the projects FSA or CMF files, Analysis Method, the Panels and Bins, etc. At this point an e-mail notification is sent to the user 1. In another embodiment the email is sent directly to the crime laboratory analyst. This electronic e-mail notification indicates that the samples 40 (project) are ready for the user 1 review.

In the event that the vendor laboratory 20 analyst still find a disagreement or an anomaly after the minor corrective action then a major corrective action at the vendor laboratory 20 will be implemented. A major corrective action is defined as is a sample 40 that needs to be re-extracted or re-isolated. The sample 40 is retested by the vendor laboratory 20 according to the LIMS 24 template, which dictates the proper major corrective action. The samples 40 are added to the vendor laboratory 20 worklist for the appropriate instrumentation at the appropriate production area. The original samples 40 and corresponding information (FSA or CMF files, Controls, ALLELIC LADDERS, Analysis Method, Panels and Bins, etc.) are linked to the retested sample 40 (FSA or CMF files, Controls, ALLELIC LADDERS, Analysis Method, Panels and Bins, etc.). Two analyst independently review samples 40 on the vendor secure Web or Internet enabled servers. Utilizing the web/Internet enabled servers the analyst confirms the Allele Ladders, Isolation Positive Controls, Isolation Negative Controls, Amplification Positive Controls, and the Amplification Negative Controls. Further, the vendor Web/Internet enabled servers allow for the vendor analyst to generate the appropriate allele calls for the samples 40. Notes from the vendor laboratory analyst are recorded on the servers. After the samples 40 are analyzed with the proper controls the vendor laboratory 20 analyst passes the samples 40 or fails the samples 40. In the event that the vendor laboratory 20 analyst passes the samples 40, the original sample 40 including the FSA or CMF files including Controls, ALLELIC LADDERS, Analysis Method, Panels and Bins, etc. are linked to the retested sample 40 including the FSA or CMF files including Controls, ALLELIC LADDERS, Analysis Method, Panels and Bins, etc. The allele calls are then compared against the vendor laboratory 20 staff profiles. The sample 40 is then added to the user 1 account on the Web server along with all the other associated files. An e-mail notification is automatically sent to the crime analyst informing them that the profiles are on the secure server ready for their review.

If the vendor laboratory 20 analyst finds that the samples 40 are unacceptable the analyst will fail the samples 40. The original sample 40 data such as the FSA or CMF files including the Controls, ALLELIC LADDERS, the Analysis Method, the Panels and Bins, are linked to the retested sample 40 data such as the FSA or CMF files, including the Controls, ALLELIC LADDERS, the Analysis Method, the Panels and Bins, etc. If the sample 40 has been tested twice in the sample 40 may be billed. The vendor laboratory 20 generates an invoice upon notification to the user 1 that the sample 40 has failed multiple processing attempts. The vendor laboratory 20 generates an invoice upon notification to the user 1 that the sample 40 has failed multiple processing attempts.

Example 1 Human Swab Sample 40 Collection Method. A Template 30 is created by the vendor laboratory 20 and associated with a particular group of samples 40 from a user 1. The information that is captured in Template 30 may include the state or federal contract number. Additionally, the type(s) of sample 40 is also recorded (i.e. All Arrestee, Convicted Offender, Paternity or Casework). The Template 30 may include the number of samples 40 that will be submitted under a particular contract. The Template 30 will dictate the autosomal (including high-resolution autosomal kits for degraded DNA or low copy number such as APPLIED BIOSYSTEM (Foster City, Calif.) MINIFILER kit), mitochondrial or Y chromosome STR kit type that is to be used on these particular sample and recorded in LIMS 24. The electrophoresis platform such as the 3100 series will be outlined in the Template 30. There are a number of thresholds that are also characterized in the Template 30. These thresholds include the raw peak height, the peak height ratio cut off, the stutter ratio cut off, the negative adenylation cut off (−A) and the percent shoulder cut off. Other technical parameters that are detailed in the Template 30 include how to process tri-alleles and off ladder alleles. The Template 30 will record the spiking and enriching policy for each sample 40 associated with this template. The reaction volume and limitations will also be outlined and recorded in the Template 30. The control sample 40 order will also be dictated by the contract and recorded. For users 1 requiring PROFILER/COFILER the Template 30 will define the number of samples 40 to be punched per profile for blood cards.

The Template 30 will define the length of performance and be recorded. The Template 30 will record the turnaround time necessary. Additionally, the Template 30 will dictate how many samples 40 are to be released on a specific timeline. The Template 30 will define to the possibility of composite profiling. Moreover, the template 30 defines the sample 40 return and or destruction policies of the original sample 40 as well as the isolated DNA and the amplified DNA. The Template 30 will also dictate how much of the sample 40 the vendor lab can use when performing screening. The Template 30 the Internal Lane Standard sizing range. The Template 30 will also record if a particular sample 40 cannot be processed on the same plate as another sample 40. Additional information recorded into the LIMS 24 Template 30 that is not mentioned here.

The next step in the process is that the vendor laboratory 20 will in many instances send prepaid overnight carriers (FedEx, UPS or DHL) labels. The overnight carriers shipping label barcodes (i.e. transportation packaging identifier) are recorded in the vendor laboratories LIMS 24 and affiliated with the contract numbers. In this example the remote user provided the genetic profile/line identification which is associated with a LIMS 24 template. The line included at least one designated genetic sequence. The genetic line identification has been previously associated with the designated genetic sequence that includes microsatellites such as D8S1179, D21S11, D7S820, CSF1P0, D3S1358, TH01, D13S317, D16S539, D2S1338, D19S433, vWA, TPOX, D18S51, Amelogenin, D5S818, and FGA. These microsatellites are included in the AMPF STR IDENTIFIER PCR Amplifications Kit (APPLIED BIOSYSTEMS, Foster City, Calif.). The transportation identifier, the sample 40 identifier and at least one genetic sequence identifier are stored in a database in association with the template 30.

PURITAN (Guilford, Me.) Cotton Swabs, with biomatter adhered thereto were used to collect DNA samples 40 from the oral cavities of three humans. The samples 40 were given a Sample 40 Identification. The samples 40 were placed into a barcoded multi-well container. The Sample 40 Identification or sample 40 identifier was electronically associated with the multi-well container in the LIMS 24 for tracking for chain-of-custody purposes. Each subsequent transfer, location and results was recorded in the LIMS 24.

The three biological samples 40 in the form of room temperature swabs were submitted via FEDEX (Memphis, Tenn.) overnight delivery to the vendor laboratory 20 from the remote user. Each sample 40 occupies one well of a 96-well multi-well container. A lysis reagent such NUCLEI LYSING SOLUTION purchased from (PROMEGA CORP., Madison, Wis. (A7943) or a created Lysis Solution such as 1% Triton X-100, 1% 5.0M NaCl, 2% 0.5M EDTA, 2% Proteinase K and 1% 1.0M Tris-HCl (pH 7.5) was gently poured into a trough or reservoir and was placed on the deck of a TECAN GENESIS WORKSTATION (Research Triangle Park, N.C.). An automated workstation 14 which is a series of apparatus communicatively coupled to the LIMS 24.

The liquid handler dispensed 500 μl of the lysis reagent in to each sample 40 well of the multi-well container. The multi-well was sealed, incubated at 55C for three hours and placed on a vortex for Iminute. The well multi-well was then placed back on the deck of the TECAN GENESIS WORKSTATION (Research Triangle Park, NC). The liquid handler aspirated 50 μl of each sample 40 and dispenses it in to a 384 well primary master well container (Fisher Scientific #NC9134044). Once all of the samples 40 are transferred, the primary master well container is moved to the deck of the Isolation Station 58.

One-hundred and twelve microliters of SV LYSIS reagent (PROMEGA CORP., Madison Wis., # Z305X) a chaotropic salt solution was added to each sample 40. Next, 10 μl of magnetic particles (PROMEGA CORP, #A220X) are added and the well components are mixed. The well plate is then moved into a magnetic field where the magnetic particles were drawn to the bottom of each well. The supernatant was then aspirated and discarded. The well plate was moved out of the magnetic field and 95 ∥l of SV LYSIS reagent was added to each well and mixed. The well plate is then moved into the magnetic field and the supernatant was drawn off and discarded. This washing process was repeated two additional times. Next, the samples 40 were washed four times in 130 μl of 95% ethanol as described above. After the fourth ethanol wash, the microwell container are placed on a 384 tip dryer for 11 minutes. Then the microwell container was moved back to the deck of the Isolation Station 58 and 155 μl of AMBION'S (Houston, Tex.) nuclease free water (catalog #B9934) was added to each well at room temperature. In lieu of utilizing the 384 well optical storage plate (Fisher Scientific, #08-772136) for optical density analysis, a bar-coded Quantification Station 60 384 well plate was created. This quantification methodology used Real Time PCR to quantify the amount concentration of DNA from the isolation process.

The plate was then moved into the magnetic field and 2 μl of DNA elution was transferred to a barcoded Quantification Station 60 384 well plate using the TECAN GENESIS WORKSTATION. Real time PCR was done with Human telomerase primers and standards to quantify the DNA in each sample 40. The 2 μl of DNA from the elution was mixed with 23 ul of reaction mix containing 10.5 μl of primer and 12.5 μl of reaction buffer with tag and IPC internal control components using the. TECAN GENESIS WORKSTATION. Serial dilutions of the standards ranging from 50 ng/μl to 0.02 ng/μl were prepared as per APPLIED BIOSYSTEM (Foster City, Calif.) QUANTIFILER protocol using the. TECAN GENESIS WORKSTATION. The samples 40 were amplified simultaneously with unknown samples 40 using the APPLIED BIOSYSTEM 7900 Real Time PCR Instrument. The DNA quantity was calculated by the software using the standard graph.

Based on the resulting yields of DNA, as determined by quantification, the TECAN GENESIS WORKSTATION would dilute (normalize) each sample 40 that exceeds the predetermined concentration with an aqueous solution such as water or Tris-EDTA (TE). The primary master well plate with the isolated DNA is moved to the deck of a TECAN GENESIS WORKSTATION. The AMPF STR IDENTIFILER PCR Amplifications Kit components and AMBION water were placed on the deck as well. The final PCR mixture is made of 1×AMPF STR PCR Master Mix, 1×PCR Primer Set (60%) and 40% isolated DNA. The TECAN GENESIS added the reagents together in the 384 Well PCR Plate. The plate is then sealed with optical sealing tape (ABI, #4311971). The samples 40 were then placed in a DNA ENGINE THERMOCYCLER (Bio-Rad, Hercules, Calif.). The AMPFlSTR® IDENTIFILER PCR Amplifications PCR thermal profile was performed as per the AMPF STR® IDENTIFILER PCR Amplifications Kit.

The PCR well plate with the amplified DNA was moved to the deck of a TECAN GENESIS WORKSTATION. The deionized formamide/GENESCAN 500 LIZ (PN 4322682) Internal Lane Size Standard solution were loaded onto the deck of the TECAN Workstation 14. The TECAN GENESIS added the 1.0 μl amplified PCR products to the 25 μl of formamide/Internal Lane Size Standard in a 96 Well PCR Plate, as described in the AMPFlSTR® IDENTIFILER PCR Amplifications Kit literature. Other well locations in the 96 Well PCR Plate were loaded with 25 μl of formamide/Internal Lane Size Standard.

The 384 plate was then placed into a sample 40 tray and placed on the autosampler of APPLIED BIOSYSTEM 3130XL capillary electrophoresis machine. The ABI 3130XL GENETIC ANALYZER performs the auto loading, capillary electrophoresis and data capture of the samples 40. The data was processed with an EXPERT SYSTEM. The data 70 from one sample 40 was found to be non-conforming to the LIMS 24 Template. LIMS 24 creates a dynamic automated worklist for the specific automation that is defined in the template 30 as being appropriate for the Corrective Action. In this particular example one sample 40 was found to have Off-Scale data and according to the template 30 the amplified DNA can be re-injected by the capillary electrophoresis machine for half the initial time. LIMS 24 dynamically creates the worklist that is imported into the capillary electrophoresis machine. That particular sample 40 is then re-injected and evaluated. The re-data was found to be conforming to the LIMS 24 Template 30 and complied with the non-Corrective Action data for transmission to the remote user.

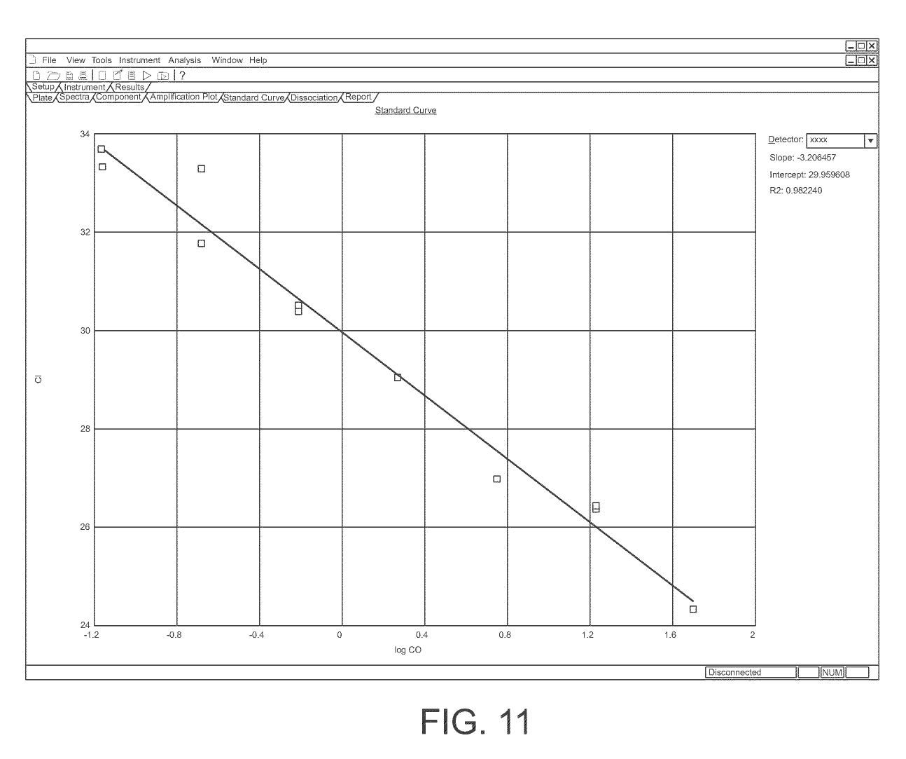

The 384 plate was then placed into a sample 40 tray and placed on the autosampler of APPLIED BIOSYSTEM 3130XL capillary electrophoresis machine. The ABI 3130XL GENETIC ANALYZER performs the auto loading, capillary electrophoresis and data capture of the samples 40. The data was processed with an EXPERT SYSTEM. On average, these results are transmitted to the remote user within twenty-four hours of receiving the biological sample 40 at the vendor laboratory 20. The results are shown in FIG. 11 and in data TABLES 1 and 2 and data are incorporated into LIMS 24.

TABLE 1
DNA Quantification from the swabs.
DNA Quantity

| | ng/μl |
|---|---|
| Swabs | 5.82 |
| Swabs | 2.83 |
| Swabs | 4.86 |

TABLE 2
STR/Microsatellite DNA Profiles from three Humans

| STR/Microsatellites | Human 1 | | Human 2 | | Human 3 | |
|---|---|---|---|---|---|---|
| AMEL | X | X | X | Y | X | X |
| CSF1PO | 10 | 12 | 11 | 12 | 10 | 12 |
| D13S317 | 12 | 13 | 8 | 11 | 12 | 13 |
| D16S539 | 12 | 9 | 9 | 10 | 12 | 13 |
| D18S51 | 12 | 18 | 13 | 17 | 14 | 18 |
| D19S433 | 12 | 13 | 14 | 14 | 12 | 12 |
| D21S11 | 30 | 32 | 29 | 31 | 29 | 32.2 |
| D2S1338 | 20 | 20 | 17 | 24 | 17 | 20 |
| D3S1358 | 16 | 17 | 16 | 17 | 15 | 17 |
| D5S818 | 12 | 12 | 12 | 13 | 12 | 13 |
| D7S820 | 8 | 10 | 11 | 8 | 8 | 12 |
| D8S1179 | 13 | 10 | 11 | 14 | 12 | 13 |
| FGA | 20 | 22 | 21 | 23 | 21 | 22 |
| TH01 | 7 | 8 | 7 | 9 | 6 | 7 |
| TPOX | 8 | 11 | 8 | 12 | 8 | 8 |
| vWA | 14 | 17 | 17 | 18 | 14 | 18 |

### Example 2

### Human Blood Card Sample Collection Method

The user 1 provided the genetic profile/line identification. The line included at least one designated genetic sequence. The genetic line identification has been previously associated with the designated genetic sequence that includes microsatellites such as D8S1179, D2151, D7S820, CSF1P0, D3S1358, TH01, D13S317, D16S539, D2S1338, D19S433, vWA, TPOX, D18S51, Amelogenin, D5S818, and FGA. These microsatellites are included in the AMPFlSTR IDENTIFILER PCR AMPLIFICATIONS KIT (APPLIED BIOSYSTEMs, Foster City, Calif.).

FTA cards (Whatman, Piscataway, N.J.), were used to collect blood samples 40. The samples 40 were given a Sample 40 Identification (“sample 40 identifier”). The samples 40 were punched and the 2 mm punched sample 40 was placed into a barcoded multi-well container. The Sample 40 Identification was electronically associated with the multi-well container in the LIMS 24 for tracking for chain-of-custody purposes. Each subsequent transfer, location and results was recorded in the LIMS 24.

The three biological samples 40 in the form of room temperature blood cards were submitted via FEDEX (Memphis, Tenn.) overnight delivery to the vendor laboratory 20 from the remote user. Each sample 40 occupies one well of a 96-well multi-well container.

A lysis reagent such NUCLEI LYSING SOLUTION purchased from (PROMEGA Corporation, Madison, Wis. (A7943) or a created Lysis Solution such as 1% Triton X-100, 1% 5.0M NaCl, 2% 0.5M EDTA, 2% Proteinase K and 1% 1.0M Tris-HCl (pH 7.5) was gently poured into a trough or reservoir and was placed on the deck of a TECAN GENESIS WORKSTATION (Research Triangle Park, N.C.). The liquid handler dispensed 100 μl of the lysis reagent in to each sample 40 well of the multi-well container. The multi-well was sealed, incubated at 55° C. for three hours and placed on a vortex for 1 minute. The well multi-well was then placed back on the deck of the TECAN GENESIS WORKSTATION (Research Triangle Park, N.C.). The liquid handler aspirated 50 μl of each sample 40 and dispenses it in to a 384 well primary master well container (Fisher Scientific #NC9134044). Once all of the samples 40 are transferred, the primary master well container is moved to the deck of the Isolation Station 58.

One-hundred and twelve microliters of SV LYSIS reagent (PROMEGA Corporation, Madison Wis., # Z305X) a chaotropic salt solution was added to each sample 40. Next, 10 μl of magnetic particles (PROMEGA Corporation, #A220X) are added and the well components are mixed. The well plate is then moved into a magnetic field where the magnetic particles were drawn to the bottom of each well. The supernatant was then aspirated and discarded. The well plate was moved out of the magnetic field and 95 μl of SV LYSIS reagent was added to each well and mixed. The well plate is then moved into the magnetic field and the supernatant was drawn off and discarded. This washing process was repeated two additional times. Next, the samples 40 were washed four times in 130 μl of 95% ethanol as described above. After the fourth ethanol wash, the microwell container are placed on a 384 tip dryer for 11 minutes. Then the microwell container was moved back to the deck of the Isolation Station 58 and 155 μl of AMBION's (Houston, Tex.) nuclease free water (catalog #B9934) was added to each well at room temperature. In lieu of utilizing the 384 well optical storage plate (Fisher Scientific, #08-772136) for optical density analysis, a barcoded Quantification Station 60 384 well plate was created. This quantification methodology used Real Time PCR to quantify the amount concentration of DNA from the isolation process.

The plate was then moved into the magnetic field and 2 μl of DNA elution was transferred to a barcoded Quantification Station 60 384 well plate using the TECAN GENESIS WORKSTATION. Real time PCR was done with Human telomerase primers and standards to quantify the DNA in each sample 40. The 2 μl of DNA from the elution was mixed with 23 ul of reaction mix containing 10.5 μl of primer and 12.5 μl of reaction buffer with tag and IPC internal control components using the TECAN GENESIS WORKSTATION. Serial dilutions of the standards ranging from 50 ng/μl to 0.02 ng/μl were prepared as per APPLIED BIOSYSTEMs (Foster City, Calif.) QUANTIFILER protocol using the TECAN GENESIS WORKSTATION. The samples 40 were amplified simultaneously with unknown samples 40 using the APPLIED BIOSYSTEMs 7900 Real Time PCR Instrument. The DNA quantity was calculated by the software using the standard graph. Based on the resulting yields of DNA, as determined by quantification, the TECAN GENESIS WORKSTATION would dilute (normalize) each sample 40 that exceeds the predetermined concentration with an aqueous solution such as water or Tris-EDTA (TE).

The primary master well plate with the isolated DNA is moved to the deck of a TECAN FREEDOM WORKSTATION 14. The AMPFlSTR IDENTIFILER PCR Amplifications Kit components and AMBION water were placed on the deck as well. The final PCR mixture is made of 1×AMPFlSTR PCR Master Mix, 1×AMPFlSTR IDENTIFILER PCR Primer Set (60%) and 40% isolated DNA. The TECAN GENESIS added the reagents together in the 384 Well PCR Plate. The plate is then sealed with optical sealing tape (ABI, #4311971). The samples 40 were then placed in a DNA ENGINE THERMOCYCLER (BIO-RAD, HERCULES, CA). The AMPFlSTR IDENTIFILER PCR Amplifications PCR thermal profile was performed as per the AMPFlSTR IDENTIFILER PCR Amplifications Kit. The PCR well plate with the amplified DNA was moved to the deck of a TECAN GENESIS WORKSTATION. The deionized formamide/GENESCAN 500 LIZ (PN 4322682) Internal Lane Size Standard solution were loaded onto the deck of the TECAN GENESIS WORKSTATION. The TECAN GENESIS added the 1.0 μl amplified PCR products to the 25 μl of formamide/Internal Lane Size Standard in a 96 Well PCR Plate, as described in the AMPF STR IDENTIFILER PCR Amplifications Kit literature. Other well locations in the 96 Well PCR Plate were loaded with 25 μl of formamide/Internal Lane Size Standard.

The 384 plate was then placed into a sample 40 tray and placed on the autosampler of APPLIED BIOSYSTEM's 3130XL capillary electrophoresis machine. The ABI 3130XL GENETIC ANALYZER performs the auto loading, capillary electrophoresis and data capture of the samples 40. The data was processed with an EXPERT SYSTEM. On average, these results are transmitted to the remote user within twenty-four hours of receiving the biological sample 40 at the vendor laboratory 20. The data are shown in TABLES 3 and 4 and are incorporated into LIMS 24.

TABLE 3
DNA Quantification from the blood cards.
DNA Quantity

| | ng/μl |
|---|---|
| Blood Card | 0.74 |
| Blood Card | 0.66 |
| Blood Card | 0.67 |

TABLE 4
STR/Microsatellite DNA Profiles from three Humans

| STR/Microsatellites | Human 1 | | Human 2 | | Human 3 | |
|---|---|---|---|---|---|---|
| AMEL | X | X | X | Y | X | X |
| CSF1PO | 11 | 12 | 10 | 12 | 10 | 12 |
| D13S317 | 12 | 13 | 8 | 11 | 9 | 12 |
| D16S539 | 11 | 9 | 9 | 10 | 12 | 13 |
| D18S51 | 12 | 18 | 13 | 17 | 14 | 18 |
| D19S433 | 12 | 13 | 14 | 14 | 12 | 12 |
| D21S11 | 29 | 32 | 29 | 31 | 29 | 30 |
| D2S1338 | 20 | 22 | 20 | 24 | 19 | 20 |
| D3S1358 | 16 | 17 | 16 | 17 | 15 | 17 |
| D5S818 | 12 | 12 | 12 | 13 | 12 | 13 |
| D7S820 | 9 | 10 | 11 | 8 | 9 | 12 |
| D8S1179 | 10 | 10 | 11 | 14 | 12 | 13 |
| FGA | 20 | 22 | 21 | 23 | 21 | 22 |
| TH01 | 7 | 8 | 7 | 9 | 6 | 7 |
| TPOX | 8 | 11 | 8 | 12 | 8 | 8 |
| vWA | 14 | 17 | 17 | 18 | 14 | 18 |

Example 3: Blood samples were collected from of three humans in vacutainers that contained EDTA as the anticoagulant. The samples were given a Sample Identification (“sample identifier”). The samples tubes were barcoded. The Sample Identification was electronically associated in the Information Management System for tracking for chain-of-custody purposes. Each subsequent transfer, location and results was recorded in the Information Management System. The three biological samples in the form of room temperature blood tubes were submitted via FEDEX (Memphis, Tenn.) overnight delivery to the vendor laboratory from the remote user.

Blood samples were collected from of three humans in vacutainers that contained EDTA as the anticoagulant. The samples were given a Sample Identification. The samples tubes were barcoded. The Sample Identification was electronically associated in the Information Management System for tracing for chain-of-custody purposes. Each subsequent transfer, location and results was recorded in the Information Management System. The thee biological samples in the form of room temperature blood tubes were submitted via FEDEX (Memphis, Tenn.) overnight delivery to the vendor laboratory from the remote user.

Each tube was placed in a rack on the TECAN GENESIS WORKSTATION (Research Triangle Park, N.C.). Each tube's barcode was scanned and associated with the rack and the position. The TECAN GENESIS WORKSTATION pierced the septum of the tubes and aspirated 250 μl of blood from each sample and placed it into a barcoded multi-well container.

A lysis reagent such NUCLEI LYSING SOLUTION purchased from (PROMEGA Corporation, Madison, Wis. (A7943) or a created Lysis Solution such as 1% Triton X-100, 1% 5.0M NaCl, 2% 0.5M EDTA, 2% Proteinase K and 1% 1.0M Tris-HCl (pH 7.5) was gently poured into a trough or reservoir and was placed on the deck of a TECAN GENESIS WORKSTATION (Research Triangle Park, N.C.). The liquid handler dispensed 300 μl of the lysis reagent in to each sample well of the multi-well container. The multi-well was sealed, incubated at 55C for three hours and placed on a vortex for 1 minute. The multi-well container was then placed back on the deck of the TECAN GENESIS WORKSTATION (Research Triangle Park, N.C.). The liquid handler aspirated 50 μof each sample and dispenses it in to a 384 well primary master well container (Fisher Scientific #N.C.9134044). Once all of the samples are transferred, the primary master well container is moved to the deck of the Isolation Station Purification Station.

One-hundred and twelve microliters of SV LYSIS reagent (PROMEGA Corporation, Madison Wis., # Z305X) a chaotropic salt solution was added to each sample. Next, 10 μl of magnetic particles (PROMEGA Corporation, #A220X) are added and the well components are mixed. The well plate is then moved into a magnetic field where the magnetic particles were drawn to the bottom of each well. The supernatant was then aspirated and discarded. The well plate was moved out of the magnetic field and 95 μl of SV LYSIS reagent was added to each well and mixed. The well plate is then moved into the magnetic field and the supernatant was drawn off and discarded. This washing process was repeated two additional times. Next, the samples were washed four times in 130 μl of 95% ethanol as described above. After the fourth ethanol wash, the microwell container are placed on a 384 tip dryer for 11 minutes. Then the microwell container was moved back to the deck of the Isolation Station Purification Station and 155 μl of AMBION's (Houston, Tex.) nuclease free water (catalog #B9934) was added to each well at room temperature.

In lieu of utilizing the 384 well optical storage plate (Fisher Scientific, #08-772136) for optical density analysis, a barcoded Quantification 384 well plate was created. This quantification methodology used Real Time PCR to quantify the amount concentration of DNA from the isolation process.

The plate was then moved into the magnetic field and 2 μl of DNA elution was transferred to a barcoded Quantification 384 well plate using the TECAN GENESIS WORKSTATION. Real time PCR was done with Human telomerase primers and standards to quantify the DNA in each sample. The 2 μl of DNA from the elution was mixed with 23p of reaction mix containing 10.5 μl of primer and 12.5 μl of reaction buffer with tag and IPC internal control components using the TECAN GENESIS WORKSTATION. Serial dilutions of the standards ranging from 50 ng/μl to 0.02 ng/μl were prepared as per APPLIED BIOSYSTEMs (Foster City, Calif.) QUANTIFILER protocol using the TECAN GENESIS WORKSTATION. The samples were amplified simultaneously with unknown samples using the APPLIED BIOSYSTEMs 7900 Real Time PCR Instrument. The DNA quantity was calculated by the software using the standard graph.

Based on the resulting yields of DNA, as determined by quantification, the TECAN GENESIS WORKSTATION would dilute (normalize) each sample that exceeds the predetermined concentration with an aqueous solution such as water or Tris-EDTA (TE).

The primary master well plate with the isolated DNA is moved to the deck of a TECAN FREEDOM WORKSTATION 14. The AMPF STR IDENTIFILER PCR Amplifications Kit components and AMBION water were placed on the deck as well. The final PCR mixture is made of 1×AMPF STR PCR Master Mix, 1×AMPF STR IDENTIFILER PCR Primer Set (60%) and 40% isolated DNA. The TECAN GENESIS added the reagents together in the 384 Well PCR Plate. The plate is then sealed with optical sealing tape (ABI, #4311971).

The samples were then placed in a DNA ENGINE THERMOCYCLER (Bio-Rad, Hercules, Calif.). The AmpFlSTR Identifiler PCR Amplifications PCR thermal profile was performed as per the AMPFlSTR IDENTIFILER PCR Amplifications Kit.

The PCR well plate with the amplified DNA was moved to the deck of a TECAN GENESIS WORKSTATION. The deionized formamide/GENESCAN 500 LIZ® (PN 4322682) Internal Lane Size Standard solution were loaded onto the deck of the TECAN WORKSTATION 14. The TECAN GENESIS added the 1.0 μl amplified PCR products to the 25 μl of formanide/Internal Lane Size Standard in a 96 Well PCR Plate, as described in the AMPFlSTR IDENTIFILER PCR Amplifications Kit literature. Other well locations in the 96 Well PCR Plate were loaded with 25 μl of formamide/Internal Standard.

The 384 plate was then placed into a sample tray and placed on the autosampler of APPLIED BIOSYSTEM's 3130XL capillary electrophoresis machine. The ABI 3130XL GENETIC ANALYZER performs the auto loading, capillary electrophoresis and data capture of the samples. The data was processed with an EXPERT SYSTEM. On average, these results are transmitted to the remote user within twenty-four hours of receiving the biological sample at the vendor laboratory. The results are shown in Tables 5 and 6.

TABLE 5
DNA Quantification from the Blood
DNA Quantity

| | ng/μl |
|---|---|
| Blood | 1.04 |
| Blood | 1.32 |
| Blood | 0.973 |

TABLE 6
STR/Microsatellite DNA Profiles from three Humans

| STR/Microsatellites | Human 1 | | Human 2 | | Human 3 | |
|---|---|---|---|---|---|---|
| AMEL | X | X | X | Y | X | X |
| CSF1PO | 11 | 12 | 10 | 12 | 10 | 12 |
| D13S317 | 12 | 13 | 8 | 11 | 9 | 12 |
| D16S539 | 11 | 9 | 9 | 10 | 12 | 13 |
| D18S51 | 12 | 18 | 13 | 17 | 14 | 18 |
| D19S433 | 12 | 13 | 14 | 14 | 12 | 12 |
| D21S11 | 29 | 32 | 29 | 31 | 29 | 30 |
| D2S1338 | 20 | 22 | 20 | 24 | 19 | 20 |
| D3S1358 | 16 | 17 | 16 | 17 | 15 | 17 |
| D5S818 | 12 | 12 | 12 | 13 | 12 | 13 |
| D7S820 | 9 | 10 | 11 | 8 | 9 | 12 |
| D8S1179 | 10 | 10 | 11 | 14 | 12 | 13 |
| FGA | 20 | 22 | 21 | 23 | 21 | 22 |
| TH01 | 7 | 8 | 7 | 9 | 6 | 7 |
| TPOX | 8 | 11 | 8 | 12 | 8 | 8 |
| vWA | 14 | 17 | 17 | 18 | 14 | 18 |

I claim:

1. A computer implemented method for processing physical crime data by a vendor laboratory for a user comprising the steps of:
receiving data to populate a template in a laboratory information management system;
receiving a transportation package identifier in computer readable form for biological samples sent by said user to said vendor laboratory;
receiving a sample identifier in computer readable form for said samples;
receiving at least one genetic sequence in computer readable form for said samples;
storing the transportation package identifier, sample identifier and at least one genetic sequence in computer readable form in the laboratory information management system in association with a template;
receiving at said vendor laboratory said samples having said transportation package identifier in computer readable form;
screening said samples in a workstation automatically directed by said template while recording said sample identifier at each step of the screening to generate physical data;
identifying at least one nonconforming sample by comparing physical data to said template;
generating a work list by said laboratory management system to rescreen said at least one nonconforming sample; and
rescreening said at least one nonconforming sample in said workstation according to the work list while recording said sample identifier at each step of the screening to generate new physical data; said workstation being communicatively coupled to said laboratory information management system.

2. The method of claim 1 further comprising the steps of:
generating electronic representations of said physical data; and
transmitting said electronic representations associated with said sample identifier to said user.

3. The method of claim 1, wherein said samples are disposed in a tube, further comprising the steps of: receiving an identifier for a tube for a biological sample and storing said tube identifier in said laboratory information management system in association with said sample identifier.

4. The method of claim 1, wherein said sample are disposed in a well of a multi-well container, further comprising the steps of: acquiring an identifier for a multi-well plate and a position of a sample in a well of a multi-well plate and storing said well plate position is said laboratory information management system in association with said sample identifier.

5. The method of claim 1 further comprising: conducting a presumptive test to associate the source of said samples to provide presumptive data and storing the presumptive data in said laboratory information management system in association with said sample identifier.

6. The method of claim 1 wherein said template is generated by populating said laboratory information management system with parameters selected from the group consisting of: a unique identifier generated by said user, types of samples, sample medium, and numbers of samples.

7. The method of claim 1 wherein said template is generated by populating said laboratory information management system with parameters selected from the group consisting of: STR kit type, electrophoresis platform, raw peak height, peak height ratio cut off, stutter ratio cut off, negative adenylation cut off (−A), and percent shoulder cut off.

8. The method of claim 1 wherein said template is generated by populating said laboratory information management system with parameters selected from the group consisting of: standard operating procedure to process tri-alleles and off ladder alleles, standard operation procedure on spiking and enriching, control sample order, and reaction volume.

9. The method of claim 1 wherein said template is generated by populating said laboratory information management system with parameters comprising blood card punches per samples associated with said sample identifier.

10. The method of claim 1 wherein said template is generated by populating said laboratory information management system with parameters selected from the group consisting of: amount of time that the user anticipates sending said samples; time allotted to screen said samples; how many samples are to be released on a specific timeline; standard operation procedure on composite profiling; and return and destruction standard operation procedure for said samples.

11. The method of claim 1 wherein said template is generated by populating said laboratory information management system with parameters selected from the group consisting of: standard operation procedure to isolate DNA, standard operating procedure to amplify DNA; how much of the sample can used for screening said samples; and internal lane standard sizing range.

12. The method of claim 1 further comprising comparing said data to a negative control.

13. The method of claim 1 further comprising comparing said data to a positive control.

14. The method of claim 1 further comprising the step of adding a first lysis reagent to a first portion of said samples associated with said sample identifier and a second lysis reagent to a second portion of said samples associated with said sample identifier.

15. An analysis laboratory with a device for receiving electronic data from a sender laboratory which is at a location which is physically separate from the analysis laboratory, with a device for receiving a physical sample which corresponds to the electronic data, the analysis laboratory comprising a template in a laboratory information management system and process controller 26 device which uses at least part of the received electronic data for automatically controlling the handling of the samples by operating units of a workstation device, the analysis laboratory further generating measurement data by evaluating the physical samples in at least one operating unit of the workstation device said laboratory information management system configured to identify data nonconforming to the template and signal the process controller for a specific portion of the workstation device to generate new measurement data, and outputting the new measurement data.

16. The analysis laboratory of claim 15 further comprising a lysis station configured for differential extraction of genetic material.
